(19) United States
(12) Patent Application Publication
(10) Pub. No.: US 2026/0049106 A1
(43) Pub. Date: Feb. 19, 2026

(54) PEPTIDE COMPOSITIONS AND METHODS OF USE

(71) Applicant: ONL Therapeutics, Inc., Ann Arbor, MI (US)

(72) Inventors: Andrew J. KOCAB, Farmington Hills, MI (US); David N. ZACKS, Ann Arbor, MI (US); Alexander J. BRIDGES, Ann Arbor, MI (US)

(21) Appl. No.: 19/100,900

(22) PCT Filed: Aug. 3, 2023
(86) PCT No.: PCT/US2023/071618

§ 371 Date: Feb. 3, 2025

Related U.S. Application Data
(60) Provisional application No. 63/370,631, filed on Aug. 5, 2022.

(51) Int. Cl. C07K 14/00; A61K 38/00

(52) U.S. Cl. CPC C07K 14/001; A61K 38/00

(57) ABSTRACT

Provided herein are compositions including peptides, pharmaceutical preparations thereof, and methods of preventing photoreceptor death therewith and protecting of retinal cells, including, but not limited to, photoreceptors and retinal pigment epithelium, from Fas- or TRAIL-mediated apoptosis.

### CROSS REFERENCE

This application claims the benefit of U.S. Provisional Patent Application No. 63/370,631 filed Aug. 5, 2022, which is incorporated herein by reference in its entirety.

### BACKGROUND

Ocular disorders and diseases affect a substantive percentage of the world's population. The leading causes of blindness and low vision include primarily age-related eye diseases such as age-related macular degeneration, cataract, diabetic retinopathy, and glaucoma. However, more acute conditions also contribute to blindness and low vision. Treatment of eye conditions resulting in blindness or vision generally loss targets curing, as well as addressing, symptoms and progression. Treatment of chronic eye conditions further aims to prevent or delay the onset of irreversible vision impairment.

Ocular inflammation and its related complications are causes of vision loss. Vision is dependent on maintaining the integrity of the structure of the retina, and changes in retinal homeostasis resulting from retinal inflammation may provide the basis for vision loss and/or retinal disease. A number of conditions such as aging, metabolic abnormalities, altered vascular perfusion, or degenerative genetic conditions may also initiate various inflammatory processes within the retina. In the case of retinal inflammation, a dysregulated and/or prolonged immune response may contribute to both the pathogenesis of retinal diseases as well as vision threatening symptoms.

Fas (CD95/APO-1) and its specific ligand (FASL/CD95L) are members of the tumor necrosis factor (TNF) receptor (TNF-R) and TNF families of proteins, respectively.

Interaction between Fas and FASL triggers a cascade of subcellular events that results in a definable cell death process in Fas-expressing targets. Fas is a 45 kDa type I membrane protein expressed constitutively in various tissues, including spleen, lymph nodes, liver, lung, kidney and ovary. (Leithauser, F. et al, Lab Invest, 69:415-429 (1993); Watanabe-Fukunaga, R. et al, J Immunol, 148:1274-1279 (1992)). FASL is a 40 kDa type II membrane protein, and its expression is predominantly restricted to lymphoid organs and perhaps certain immune-privileged tissues. (Suda, T. et al, Cell, 75:1169-1178 (1993); Suda, T. et al, J Immunol, 154:3806-3813 (1995)). In humans, FASL can induce cytolysis of FAS-expressing cells, either as a membrane-bound form or as a 17 kDa soluble form, which is released through metalloproteinase-mediated proteolytic shedding. (Kayagaki, N. et al, J Exp Med, 182:1777-1783 (1995); Mariani, S. M. et al, Eur J Immunol, 25:2303-2307 (1995)).

Binding of Fas ligands (FasL) to Fas receptor can elicit apoptotic signals either via classical pathways or via indirect pathways (Mundle & Raza., Trends. Immuno., 23:187-194 (2002)). Independently, Fas and FasL stimulation alone can induce cell proliferation (Aggarwal et al., FEBS Lett, 364:5-8 (1995); Freiberg et al, J Invest Dermatol, 108:215-219 (1997); Jelaska & Korn, J. Cell. Physiol, 175:19-29 (1998); Suzuki et al, J Immunol, 165:5537-5543 (2000); Suzuki et al, J. Exp. Med., 187: 123-8 (1998)). Membrane bound TNF superfamily members including FasL has been show to “reverse-signal” via their membrane attach cytoplasmic tail and thus they also possess a “bi-directional” signaling (Sun & Fink, J. Immuno., 179:4307-4312 (2007)). These studies suggest that small molecules, such as Kp 7 and mimetics thereof, which bind to both Fas and FasL can regulate Fas receptor signaling in a tissue-specific manner can be used to treat a variety of autoimmune pathologies.

The FASL/FAS system has been implicated in the control of the immune response and inflammation, the response to infection, neoplasia, and death of parenchymal cells in several organs. (Nagata et al supra; Biancone, L. et al., J Exp Med, 186:147-152 (1997); Krammer, P. H. Adv Immunol, 71:163-210 (1999); Seino, K. et al, J Immunol, 161:4484-4488 (1998)). Defects of the FASL/FAS system can limit lymphocyte apoptosis and lead to lymphoproliferation and autoimmunity. A role for FASL-FAS in the pathogenesis of rheumatoid arthritis, Sjogren's syndrome, multiple sclerosis, viral hepatitis, renal injury, inflammation, aging, graft rejection, HIV infection and a host of other diseases has been proposed. (Famularo, G., et al., Med. Hypotheses, 53:50-62 (1999)). FAS mediated apoptosis is an important component of tissue specific organ damage, such as liver injury that has been shown to be induced through the engagement of the FAS-FASL receptor system. (Kakinuma, C. et al., Toxicol Pathol, 27: 412-420 (1999); Famularo, G., et al., Med Hypotheses, 53: 50-62 (1999); Martinez, O. M. et al., Int Rev Immunol, 18:527-546 (1999); Kataoka, Y. et al, Immunology, 103:310-318 (2001); Chung, C S. et al, Surgery, 130:339-345 (2001); Doughty, L. et al, Pediatr Res, 52:922-927 (2002)).

Glaucoma is an eye disorder characterized by increased pressure inside the eye (“intraocular pressure” or “IOP”), excavation of the optic nerve head and gradual loss of the visual field. An abnormally high IOP is commonly known to be detrimental to the eye, and there are clear indications that, in glaucoma patients, this probably is the most important factor causing degenerative changes in the retina. The pathophysiological mechanism of open angle glaucoma is, however, still unknown. Unless treated successfully glaucoma will lead to blindness sooner or later, its course towards that stage is typically slow with progressive loss of the vision. IOP is the fluid pressure inside the eye. Tonometry is the method eye care professionals use to determine this. IOP is an important aspect in the evaluation of patients at risk of glaucoma. Most tonometers are calibrated to measure pressure in millimeters of mercury (mmHg).

In retinal cells, Fas receptor is activated by Fas ligand (FasL). Fas mediates cell death directly via multiple pathways: extrinsic apoptosis (through caspase cascade), intrinsic apoptosis (through Bid/Bax), and necroptosis (through RIPK1/3). Fas also mediates cell death indirectly through multiple immune response pathways: inflammasome (NLRP3, IL1β, TNFα), inflammasome-independent IL1β activation, HMGB1 nuclear release and secretion, and others yet to be determined.

It has been shown that photoreceptor cells in culture are very sensitive to apoptosis induced by FasL suggesting that FasL-induced apoptosis is a major contributor to vision loss in retinal diseases. (Burton. Trans Am Ophthalmol Soc. 1982; 80:475-497; Ross et al. Ophthalmology. 1998; 105(11):2149-2153; Hassan et al. Ophthalmology. 2002; 109(1):146-152.) Furthermore, a small peptide inhibitor of the Fas receptor, Met-12, H⁶⁰HIYLGAVNYIY⁷¹ derived from the Fas-binding extracellular domain of the oncoprotein Met. (Zou et al. Nature Medicine 13, 1078 (2007) has been shown to be photoreceptor protective, both in cell culture experiments, and in the setting of separation of the retinal and retinal pigment epithelium and other ocular conditions or diseases. (Besirli et al., Invest Ophthalmol Vis Sci., 51(4):2177-84 (2010); U.S. Pat. No. 8,343,931; herein incorporated by reference in their entireties). Furthermore c-Met, presumably using the same binding domain with homology to Met-12, FasL, TNAα and TRAIL has been shown to block TRAIL-induced apoptosis in various tumors. (Du et al. PLoS One 9, e95490 (2014))

The Met-12 peptide itself has biopharmaceutical properties, dominated by its extremely poor aqueous solubility. Experiments have clearly shown that Met-12 has to be dosed as a solution, both in vitro and in vivo, to show optimal activity, and producing such solutions in a largely aqueous medium has proven to be very difficult, especially under conditions which are acceptable for intravitreal injection. Dosing of suspensions or gels of Met-12 leads to major losses of potency. For example, even an apparently clear 10 rang/mL solution of Met-12 in 20 mM citrate buffer pH 2.8 showed a considerable loss of material upon filtration, and when used in both the in vitro and in vivo assays described below, led to at least a fivefold loss in activity. Despite extensive development work, the only solution formulations of Met-12 which have been found involve some very low pH solution injections (<pH 2.8) or neat DMSO injections, all of which are suboptimal for intravitreal injections.

As such, there still exists a need for developing Fas inhibitors, compositions including Fas inhibitors, and methods of using the Fas inhibitors in order to prevent or ameliorate various diseases or conditions.

### SUMMARY

Despite advances in therapeutics aimed at treating ocular disorders and preserving or improving vision, the burden resulting from ocular diseases and disorders continues to increase. Many challenges exist in the development of therapeutics for treating disease and disorders in the eye of an individual.

In one aspect, the present disclosure provides an oligopeptide or peptidomimetic of Formula (Ia) or Formula (Ib):

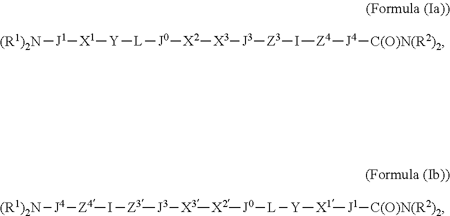

or a pharmaceutically acceptable salt thereof, wherein:
J⁰ is a linker region consisting of two to five amino acid residues;
J¹ and J⁴ are each optional linker regions consisting of one to six amino acid residues;
J³ is an optional linker region consisting of one to three amino acid residues;
Z³, Z⁴, Z^{3′}, and Z^{4′} are each an amino acid residue selected from histidine (H), tyrosine (Y), phenylalanine (F), and tryptophan (W);
X¹ and X^{1′} are each a tripeptide region consisting of three amino acid residues, wherein at least one amino acid residue is selected from histidine (H), lysine (K), arginine (R), alanine (A), cysteine (C), and methionine (M), and one amino acid residue is selected from leucine (L), isoleucine (I), methionine (M), and valine (V);
X² and X^{2′} are each independently selected from (i) and (ii):
   (i) when Z³ and Z⁴ are each tyrosine (Y) or when Z³ and Z⁴ are each tyrosine (Y), X² and X^{2′} are each selected from leucine (L), isoleucine (I), alloisoleucine, methionine (M), serine (S), and threonine (T); or
   (ii) when at least one of Z³ and Z⁴ is not tyrosine (Y) or when at least one of Z³ and Z⁴ is not tyrosine (Y),
      X² and X^{2′} are each selected from glycine (G), alanine (A), valine (V), leucine (L), isoleucine (I), alloisoleucine, methionine (M), serine (S), threonine (T);
X³ and X^{3′} are each an amino acid residue selected from asparagine (N), glutamine (Q), serine (S), and threonine (T);
R¹ is selected at each occurrence from hydrogen, —C(O)C₁₋₄ alkyl, and B—R³;
R² is selected at each occurrence from hydrogen and B—R³;
   wherein B is absent or an organic linker, and R³ is a label or a water-soluble oligomer;
wherein the amino acid residues of X¹, X², X³, Z³ and Z⁴ are L-amino acid residues, and the amino acid residues of X^{1′}, X^{2′}, X^{3′}, Z^{3′} and Z^{4′} are D-amino acid residues.

In some embodiments, the oligopeptide or peptidomimetic is represented by Formula (Ia-1) or Formula (Ib-1):

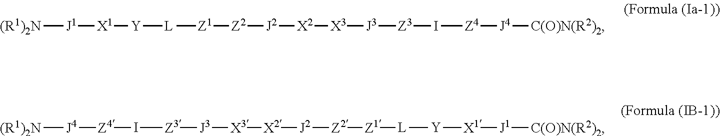

or a pharmaceutically acceptable salt thereof, wherein:
J² and J³ are each an optional linker region consisting of one to three amino acid residues, wherein J² and J³ comprise the same number of amino acid residues; and
Z¹, Z^{1′}, Z², and Z^{2′} are each independently selected from glycine (G), alanine (A), and serine (S), wherein the amino acid residues of Z¹ and Z² are L-amino acid residues, and the amino acid residues of Z^{1′} and Z²″ are D-amino acid residues.

In some embodiments, Z¹, Z^{1′}, Z², and Z^{2′} are each independently selected from glycine (G) and alanine (A).

In some embodiments, the oligopeptide or peptidomimetic is represented by Formula (Ia-2) or Formula (Ib-2):

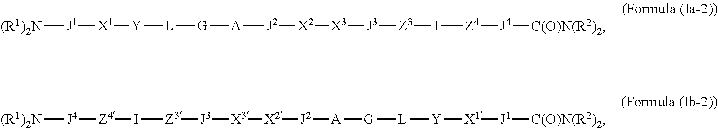

or a pharmaceutically acceptable salt thereof.

In some embodiments, Z³, Z⁴, Z^{3′}, and Z^{4′} are each an amino acid residue selected from tyrosine (Y) and phenylalanine (F).

In some embodiments, the oligopeptide or peptidomimetic is represented by Formula (Ia-3) or Formula (Ib-3):

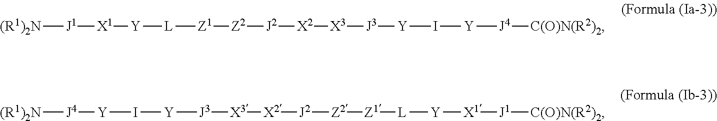

or a pharmaceutically acceptable salt thereof.

In some embodiments, wherein the oligopeptide or peptidomimetic is represented by Formula (Ia-4) or Formula (Ib-4):

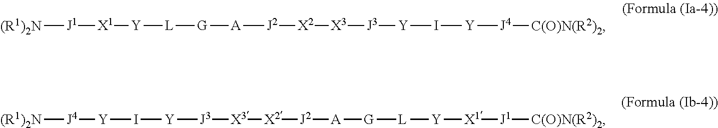

or a pharmaceutically acceptable salt thereof.

In some embodiments, at least two consecutive amino acid residues of X¹ and X^{1′} are selected from histidine (H), lysine (K), and arginine (R). In some embodiments, at least one of the amino acid residues of X¹ and X^{1′} is histidine (H). In some embodiments, X¹ and X^{1′} consist of the following amino acid sequence: —H—H—I—. In some embodiments, X² and X^{2′} are each an amino acid residue selected from glycine (G), alanine (A), valine (V), serine (S), and threonine (T). In some embodiments, X² and X^{2′} are each valine (V). In some embodiments, X² and X^{2′} are each an amino acid residue selected from glycine (G), alanine (A), serine (S), and threonine (T). In some embodiments, X³ and X^{3′} are each asparagine (N).

In some embodiments, J¹, J², J³, and J⁴ are each absent. In some embodiments, at least one of J, J², and J⁴ is a peptide linker region comprising at least one unnatural amino acid residue.

In some embodiments, each occurrence of R¹ and R² is hydrogen.

In some embodiments, the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

In some embodiments, at least one amino acid residue of Z¹, Z^{1′}, Z², Z^{2′}, Z³, Z^{3′}, Z⁴, and Z^{4′} is an unnatural amino acid residue. In some embodiments, at least one amino acid residue of Z³, Z^{3′}, Z⁴, and Z^{4′} is an unnatural amino acid residue having a side chain comprising a substituted phenyl ring.

In another aspect, the present disclosure provides an oligopeptide or peptidomimetic of Formula (IIa) or Formula (IIb):

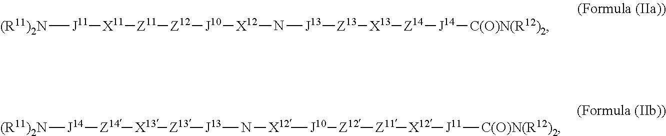

or a pharmaceutically acceptable salt thereof, wherein:
J¹⁰ is a linker region consisting of two to five amino acid residues;
J¹¹ and J¹⁴ and are each optional linker regions consisting of one to six amino acid residues;
J¹³ is an optional linker region consisting of one to three amino acid residues;
Z¹¹ and Z^{11′} are each an amino acid residue selected from: tyrosine (Y), phenylalanine (F), tryptophan (W), alanine (A), valine, (V), leucine (L), isoleucine (I), alloisoleucine, and methionine (M);
Z¹² and Z^{12′} are each an amino acid residue selected from: glycine (G), alanine (A), valine (V), leucine (L), isoleucine (I), and alloisoleucine;
Z¹³, Z¹⁴, Z^{13′}, and Z^{14′} are each an amino acid residue selected from histidine (H), tyrosine (Y) phenylalanine (F), and tryptophan (W);
X¹¹ and X^{11′} are each a tripeptide region consisting of three amino acid residues, wherein at least one amino acid residue is selected from histidine (H), lysine (K), arginine (R), alanine (A), cysteine (C), and methionine (M), and one amino acid residue is selected from leucine (L), isoleucine (I), methionine (M), and valine (V); provided that at most one of the three amino acid residues of X¹¹ and X^{11′} is histidine;
X¹² and X^{12′} are each an amino acid residue selected from glycine (G), alanine (A), valine (V), leucine (L), isoleucine (I), and methionine (M);
X¹³ and X^{13′} are each an amino acid residue selected from valine (V), leucine (L), isoleucine (I), alloisoleucine, methionine (M), phenylalanine (F), and tyrosine (Y);
R¹¹ is selected at each occurrence from hydrogen, —C(O)C₁₋₄ alkyl, and B—R¹³;
R¹² is selected at each occurrence from hydrogen and B—R¹³;
   wherein B is absent or an organic linker, and R¹³ is a label or a water-soluble oligomer;
wherein the amino acids of X¹¹, X¹², Z¹¹, Z¹², Z¹³, and Z¹⁴ are L-amino acids, and the amino acids of X^{11′}, X^{12′}, Z^{11′}, Z^{12′}, Z^{13′}, and Z^{14′} are D-amino acids.

In some embodiments, the oligopeptide or peptidomimetic is represented by Formula (IIa-1) or Formula (IIb-1):

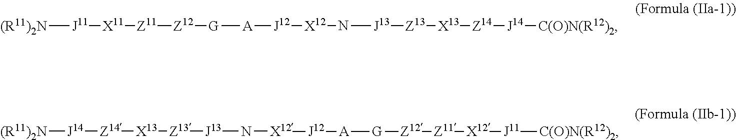

or a pharmaceutically acceptable salt thereof, wherein:
J¹² and J¹³ are each an optional linker region consisting of one to three amino acid residues, wherein J¹² and J¹³ comprise the same number of amino acid residues.

In some embodiments, Z¹¹ and Z¹¹ are each an amino acid residue selected from: tyrosine (Y), phenylalanine (F), tryptophan (W). In some embodiments, Z¹² and Z^{12′} are each leucine (L).

In some embodiments, the oligopeptide or peptidomimetic is represented by Formula (IIa-2) or Formula (IIb-2):

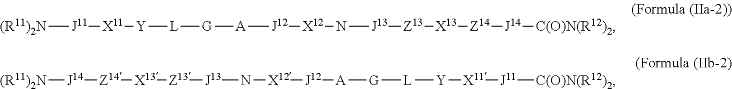

or a pharmaceutically acceptable salt thereof.

In some embodiments, Z¹³, Z¹⁴, Z^{13′}, and Z^{14′} are each an amino acid residue selected from tyrosine (Y) and phenylalanine (F).

In some embodiments, X¹³ and X^{13′} are each an amino acid residue selected from leucine (L), isoleucine (I), and alloisoleucine.

In some embodiments, the oligopeptide or peptidomimetic is represented by Formula (IIa-3 or Formula (IIb-3):

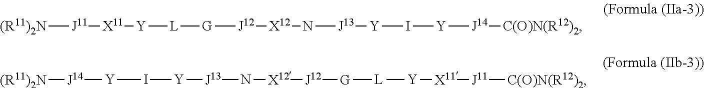

or pharmaceutically acceptable salt thereof.

In some embodiments, the oligopeptide or peptidomimetic is represented by Formula (IIa-4) or Formula (IIb-4):

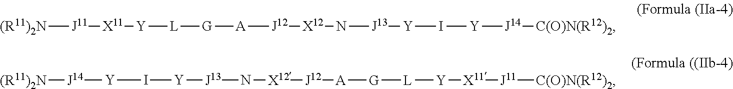

or pharmaceutically acceptable salt thereof.

In some embodiments, at least two consecutive amino acid residues of X¹¹ and X^{11′} are selected from histidine (H), lysine (K), and arginine (R), and one amino acid residue is isoleucine (I). In some embodiments, at least one of the amino acid residues of X¹ and X^{1′} is histidine (H). In some embodiments, X¹² and X^{12′} are each valine (V).

In some embodiments, J¹¹, J¹², J¹³, and J¹⁴ are each absent. In some embodiments, at least one of J¹¹, J¹², J¹³, and J¹⁴ is present.

In some embodiments, each occurrence of R¹ and R² is hydrogen.

In some embodiments, the oligopeptide or peptidomimetic comprises at least one peptidomimetic amino acid residue.

In some embodiments, at least one peptidomimentic amino acid residue are independently selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

In some embodiments, at least one amino acid residue of Z¹¹, Z^{11′}, Z¹², Z^{12′}, Z¹³, Z^{13′}, Z¹⁴, and Z^{14′} is an unnatural amino acid residue. In some embodiments, at least one amino acid residue of Z¹¹, Z^{11′}, Z¹³, Z^{13′}, Z¹⁴, and Z^{14′} is an unnatural amino acid residue having a side chain comprising a substituted phenyl ring.

In some embodiments, at least one occurrence of R¹¹ and R¹² is B—R¹³.

In another aspect, the present disclosure provides an oligopeptide or peptidomimetic of Formula (IIIa) or Formula (IIIb):

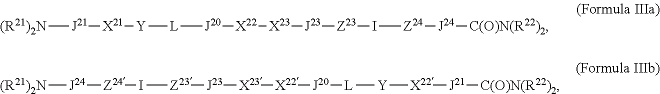

or pharmaceutically acceptable salt thereof, wherein:
J²⁰ is a linker region consisting of two to five amino acid residues;
J²¹ and J²⁴ are each optional linker regions consisting of one to six amino acid residues;
J²³ is an optional linker region consisting of one to three amino acid residues;
Z²³, Z²⁴, Z^{23′}, and Z^{24′} are each an amino acid residue selected from histidine (H), tyrosine (Y), phenylalanine (F), and tryptophan (W);
X²¹ and X^{21′} are each a tripeptide region consisting of one to six amino acid residues, each of which is independently selected from: histidine (H), lysine (K), arginine (R), alanine (A), cysteine (C), and methionine (M), and one amino acid residue is selected from leucine (L), isoleucine (I), methionine (M), and valine (V);
X²² and X^{22′} are each independently selected from glycine (G), alanine (A), valine (V), leucine (L), isoleucine (I), alloisoleucine, methionine (M), serine (S), and threonine (T);
X²³ and X^{23′} are each an amino acid residue selected from asparagine (N), glutamine (Q), serine (S), and threonine (T);
R²¹ is selected at each occurrence from hydrogen, —C(O)C₁₋₄ alkyl, and L-R²³;
R²² is selected at each occurrence from hydrogen and B—R²³, wherein at least one occurrence of R²¹ or R²² is selected from —C(O)C₁₋₄ alkyl and B—R²³;
   wherein B is absent or an organic linker, and R²³ is a label or a water-soluble oligomer; wherein the amino acid residues of X²¹, X²², X²³, Z²¹, Z²², Z²³ and Z²⁴ are L-amino acid residues, and the amino acid residues of X²¹, X²², X²³, Z^{21′}, Z^{22′}, Z^{23′} and Z^{24′} are D-amino acid residues.

In some embodiments, the oligopeptide or peptidomimetic is represented by Formula (IIIa-1) or Formula (IIIb-1):

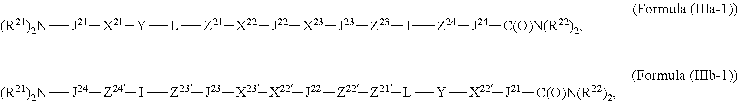

or pharmaceutically acceptable salt thereof, wherein
J²² and J²³ are each an optional linker region consisting of one to three amino acid residues, wherein J²² and J²³ comprise the same number of amino acid residues;
Z²¹, Z^{21′}, Z²² and Z^{22′} are each independently selected from: glycine (G), alanine (A), and serine (S), wherein the amino acid residues of Z²¹ and Z²² are L-amino acid residues, and the amino acid residues of Z^{21′} and Z^{22′} are D-amino acid residues.

In some embodiments, J²¹, J²², J²³, and J²⁴ are each absent. In some embodiments, at least one of J²¹, J²², J²³, and J²⁴ is a peptide linker region comprising at least one unnatural amino acid residue.

In some embodiments, the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

In some embodiments, at least one amino acid residue of Z²¹, Z^{21′}, Z²², Z^{22′}, Z²³, Z^{23′}, Z²⁴, and Z^{24′} is an unnatural amino acid residue. In some embodiments, at least one amino acid residue of Z²³, Z^{23′}, Z²⁴, and Z^{24′} is an unnatural amino acid residue having a side chain comprising a substituted phenyl ring.

In some embodiments, B is absent. In some embodiments, B is an organic linker.

In some embodiments, the organic linker of B comprises a C₁₋₁₀ alkyl chain. In some embodiments, R²³ is a label. In some embodiments, the label of R²³ comprises a fluorophore. In some embodiments, the label of R²³ comprises biotin. In some embodiments, the label of R²³ comprises an azide or an alkyne. In some embodiments, R²³ is a water-soluble oligomer. In some embodiments, the water-soluble oligomer of R²³ comprises polyethylene glycol.

In another aspect, the present disclosure provides a pharmaceutical composition comprising an oligopeptide or peptidomimetic of Formula (Ia), (Ib), (Ia-1), (Ib-1), (Ia-2), (Ib-2), (Ia-3), (Ib-3), (Ia-4), (Ib-4), (IIa), (IIb), (IIa-1), (IIb-1), (IIa-2), (IIb-2), (IIa-3), (IIb-3), (IIa-4), (IIb-4), (IIIa), (IIIb), (IIIa-1), or (IIIb-1), a pharmaceutically acceptable excipient

In another aspect, the present disclosure provides a pharmaceutical composition a method of inhibiting FasL-mediated cell death comprising administering to a subject in need thereof an oligopeptide or peptidomimetic of Formula (Ia), (Ib), (ha-1), (Ib-1), (Ia-2), (Ib-2), (Ia-3), (Ib-3), (Ia-4), (Ib-4), (IIa), (IIb), (IIa-1), (IIb-1), (IIa-2), (IIb-2), (IIa-3), (IIb-3), (IIa-4), (IIb-4), (IIIa), (IIIb), (IIIa-1), or (IIIb-1), or a pharmaceutical composition comprising an oligopeptide or peptidomimetic of Formula (Ia), (Ib), (Ia-1), (Ib-1), (Ia-2), (Ib-2), (Ia-3), (Ib-3), (Ia-4), (Ib-4), (IIa), (IIb), (IIa-1), (IIb-1), (IIa-2), (IIb-2), (IIa-3), (IIb-3), (IIa-4), (IIb-4), (IIIa), (IIIb), (IIIa-1), or (IIIb-1), and a pharmaceutically acceptable excipient.

In some embodiments, a method of preventing cell death comprising administering to a subject in need thereof an oligopeptide or peptidomimetic of Formula (Ia), (Ib), (Ia-1), (Ib-1), (Ia-2), (Ib-2), (Ia-3), (Ib-3), (Ia-4), (Ib-4), (IIa), (IIb), (IIa-1), (IIb-1), (IIa-2), (IIb-2), (IIa-3), (IIb-3), (IIa-4), (IIb-4), (IIIa), (IIIb), (IIIa-1), or (IIIb-1), or a pharmaceutical composition of Formula (Ia), (Ib), (Ia-1), (Ib-1), (Ia-2), (Ib-2), (Ia-3), (Ib-3), (Ia-4), (Ib-4), (IIa), (IIb), (IIa-1), (IIb-1), (IIa-2), (IIb-2), (IIa-3), (IIb-3), (IIa-4), (IIb-4), (IIIa), (IIIb), (IIIa-1), or (IIIb-1), pharmaceutically acceptable excipient.

### DETAILED DESCRIPTION

Provided herein are pharmaceutical preparations of biologically active, aqueous formulations of a photoreceptor-protective peptide, pharmaceutical preparations thereof, and methods of preventing photoreceptor death therewith as well as therapeutic methods.

### Definitions

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, compositions, devices and materials are described herein.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The terms “optional” or “optionally” mean that the subsequently described event, circumstance, or component may but need not occur, and that the description includes instances where the event or circumstance occurs and instances in which it does not.

The terms “peptide”, “oligopeptide,” or “polypeptide” are used interchangeably in the context of the present invention to refer to a chain of at least two amino acids linked by peptide bonds. Oligopeptides can include amino acid residues including natural and/or non-natural amino acid residues. In some embodiments, the term “oligopeptide” in the context of the present invention is used to refer to amino acid chains with, e.g., between 7 and 50 amino acid residues, between 8 and 30 amino acid residues, between 8 and 20 amino acid residues, between 8 and 25 amino acid residues, or between 9 and 15 amino acid residues.

In certain embodiments, the oligopeptide or peptidomimetic comprises one or more naturally occurring amino acids. In certain embodiments, the oligopeptide or peptidomimetic consists of naturally occurring amino acids. As used herein, naturally occurring amino acids include and/or refer to amino acids which are generally found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr). In some embodiments, the oligopeptide or peptidomimetic of the present disclosure comprises a C-terminal amide (i.e., the oligopeptide or peptidomimetic is C-terminally amidated).

As used herein, the structure of the oligopeptides and the peptidomimetics are represented in the following format:

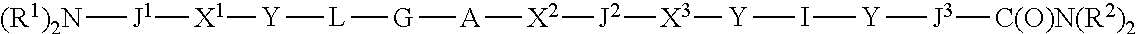

In this format, amino acid residues are referred to by their single letter name (e.g., K for lysine, F for phenylalanine, Y for tyrosine, etc.). Variable regions are represented by the letters X, Z, and J. For example, each X and Z represents a variable position consisting of a single amino acid residue. Each J represents an optional variable region comprising one or more variable amino acid residues. The covalent connection between each amino acid residue is represented by “-”. In the case of an oligopeptide, “-” represents an amide bond between two residues. In the case of a peptidomimetic having one or more peptide isosteres in the peptide backbone, “-” represents either an amide bond between two residues or a peptide isostere (e.g., a peptoid amino acid residue). In this format, the amino group on the N-terminus is represented by “(R)₂N—,” which is substituted with two R groups. The C-terminus is represented by —“C(O)OR” or “—C(O)N(R)₂”. For oligopeptides or peptidomimetics lacking a C-terminal amide, “—C(O)OR” represents the carboxyl group at the C-terminus of the oligopeptide or peptidomimetic, which is substituted with one R group. For oligopeptides or peptidomimetics having a C-terminal amide, “—C(O)N(R)₂” represents the amide group at the C-terminus of a C-terminally amidated oligopeptide or peptidomimetic, which is substituted with two R groups.

Thus, for example, Compound A, shown below may be represented by the following formula:

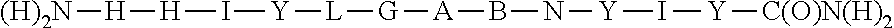

For comparison, the chemical structure of Compound A is provided below:

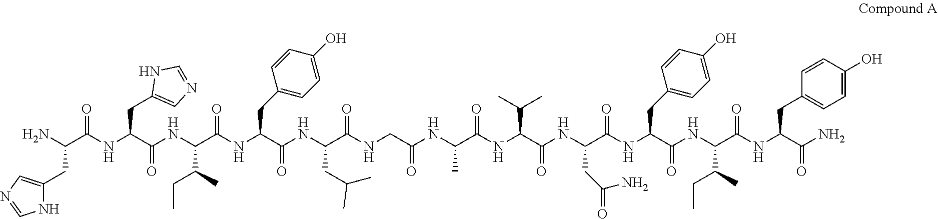 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

In some embodiments, the oligopeptide or peptidomimetic comprises a variant sequence of the oligopeptide or peptidomimetic. In certain instances, amino acid substitutions can be made in the sequence of any of the oligopeptide or peptidomimetic described herein, without necessarily decreasing or ablating its activity (as measured by, e.g., the binding or functional assays described herein). Accordingly, in some embodiments, the variant sequence comprises one or more amino acid substitutions. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine and histidine. In certain instances, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids.

Peptidomimetic compositions can contain any combination of non-natural structural components, which are typically from three structural groups: (a) residue linkage groups other than the natural amide bond (“peptide bond”) linkages; (b) non-natural residues in place of naturally occurring amino acid residues; or (c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Linking groups that can be an alternative to the traditional amide bond (“peptide bond”) linkages include, e.g., ketomethylene (e.g., —C(═O)—CH₂— for C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola, 1983, Chemistry and Biochemistry of Amino Acids, Peptides and Proteins 7:267-357).

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl)carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, guanidino-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form 0-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a Bcl polypeptide having biological activity of the invention, resulting in modification of the structure of the polypeptide, without significantly altering its biological activity. For example, amino- or carboxyl-terminal, or internal, amino acids that are not required for biological activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9:205-223, 1998; Hruby, Curr. Opin. Chem. Biol. 1:114-119, 1997; Ostergaard, Mol. Divers. 3:17-27, 1997; Ostresh, Methods Enzymol. 267:220-234, 1996. Modified peptides of the invention can be further produced by chemical modification methods, see, e.g., Belousov, Nucleic Acids Res. 25:3440-3444, 1997; Frenkel, Free Radic. Biol. Med. 19:373-380, 1995; Blommers, Biochemistry 33:7886-7896, 1994.

In certain embodiments, oligopeptide or peptidomimetic comprises one or more non-natural amino acids. In certain embodiments, oligopeptide or peptidomimetic consists of non-natural amino acids. As used herein, non-natural amino acids and/or unnatural amino acids include and/or refer to amino acid structures that cannot be generated biosynthetically in any organism using unmodified or modified genes from any organism. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring oligopeptide or peptidomimetic sequence. A non-natural amino acid residue also can be incorporated such that a desired functionality is imparted to the apelin molecule, for example, the ability to link a functional moiety (e.g., PEG).

Polynucleotide and peptide sequences of the current disclosure can be defined in terms of particular identity and/or similarity with certain polynucleotides and proteins described herein. In some embodiments, sequence identity will typically be greater than 60%, greater than 75%, greater than 80%, greater than 90%, and/or greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence disclosed herein. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net). In some embodiments, BLAST searches are be performed with the BLAST program, score=100, wordlength=12, to obtain sequences with the desired percent sequence identity. In some embodiments, to obtain gapped alignments for comparison purposes, Gapped BLAST. When utilizing BLAST and Gapped BLAST programs, the default parameters of the respective programs (NBLAST and XBLAST) can be used. In some embodiments, Clustal Omega is used.

A polynucleotide sequence or nucleotide sequence or nucleic acid sequence, as used interchangeably herein, is a polymer of nucleotides, including an oligonucleotide, a DNA, and RNA, a nucleic acid, or a character string representing a nucleotide polymer, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand.

The term “reduced” or “reduce” as used herein generally means a decrease by at least 5% as compared to a reference or control level, for example, a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, or any integer decrease between 10-100% as compared to a reference or control level.

The term “increased” or “increase” as used herein generally means an increase of at least 5% as compared to a reference or control level, for example an increase of at least 10% as compared to a reference level, or at least about 20%, or at least about 30%, or at least about 40%, or at least about 50%, or at least about 60%, or at least about 70%, or at least about 80%, or at least about 90% or up to and including a 100% increase or any integer increase between 10-100% as compared to a reference level, or about a 2-fold, or about a 3-fold, or about a 4-fold, or about a 5-fold or about a 10-fold increase, or any increase between 2-fold and 10-fold or greater as compared to a reference or control level.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to the eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

“Water-soluble” as in a “water-soluble oligomer” indicates an oligomer that is at least 35% (by weight) soluble, and preferably greater than 95% soluble, in water at room temperature. On a weight basis, a “water-soluble” oligomer is preferably at least 35% (by weight) soluble in water, more preferably at least 50% (by weight) soluble in water, still more preferably at least 70% (by weight) soluble in water, and still more preferably at least 85% (by weight) soluble in water. It is most preferred, however, that the water-soluble oligomer is at least 95% (by weight) soluble in water or completely soluble in water.

An “oligomer” is a molecule possessing from about 1 to about 30 monomers. The architecture of an oligomer can vary. Specific oligomers for use in the invention include those having a variety of geometries such as linear, branched, or forked, to be described in greater detail below.

Any water-soluble oligomer can be used in embodiments of the present disclosure and the invention is not limited in this regard. In some embodiment, the water-soluble oligomer comprises an oligomer selected from the group consisting of poly(alkylene oxide), poly(vinyl pyrrolidone), poly(vinyl alcohol), polyoxazoline, poly(acryloylmorpholine), and combinations thereof. It is particularly, preferred, however, that a poly(alkylene oxide) such as a poly(ethylene glycol) (“PEG”) derivative is used as the polymer in the present invention.

“PEG” or “polyethylene glycol,” as used herein, is meant to encompass any water-soluble poly(ethylene oxide). Unless otherwise indicated, a “PEG oligomer” or an oligoethylene glycol is one in which all of the monomer subunits are ethylene oxide subunits. Typically, substantially all, or all, monomeric subunits are ethylene oxide subunits, though the oligomer may contain distinct end capping moieties or functional groups, e.g. for conjugation. Typically, PEG oligomers for use in the present invention will comprise one of the two following structures: “—(CH₂CH₂O)ₙ” or “—(CH₂CH₂O)ₙ₋₁CH₂CH₂—,” depending upon whether or not the terminal oxygen(s) has been displaced, e.g., during a synthetic transformation. As stated above, for the PEG oligomers of the invention, the variable (n) ranges from 1 to 30, and the terminal groups and architecture of the overall PEG can vary. When PEG further comprises a functional group, A, for linking to, e.g., a small molecule drug, the functional group when covalently attached to a PEG oligomer, does not result in formation of (i) an oxygen-oxygen bond (—O—O—, a peroxide linkage), or (ii) a nitrogen-oxygen bond (N—O, O—N).

As used herein, the term “organic linker” or “linker” refers to an organic tether bridging the gap between means an organic moiety that connects two parts of a compound (e.g., the N- or C-terminus of the peptide to a label or a water-soluble oligomer. Linkers typically comprise a direct bond or an atom such as oxygen or sulfur, a unit such as NH, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NH, C(O). The terms linker and spacer are used interchangeably herein. The linker can comprise any combinations of the above. In preferred embodiments, the organic linker comprises an alkylene. In preferred embodiments, the organic linker comprises a divalent 1,6-aminohexanoic acid radical (“Ahx”) having the following structure: —N(H)—(CH₂)₆—C(O)—. In some embodiments, the linker is a linear linker or a branched linker.

The term “label” as used in the context of the present invention refers to any kind of compound suitable for specific detection. In some embodiments, the term “label” and its variants, as used herein, comprises any optically detectable moiety and includes any moiety that can be detected using, for example, fluorescence, luminescence and/or phosphorescence spectroscopy, Raman scattering, or diffraction. In some embodiments, preferred labels are selected from a fluorescent dye, a radioisotope and a contrast agent.

In some embodiments, preferred labels are selected from a fluorescent dye. A contrast agent is a dye or other substance that helps to show abnormal areas inside the body. In one embodiment the term label refers to a compound that comprises a chelating agent which forms a complex with divalent or trivalent metal cations. Preferred fluorescent dyes are selected from the following classes of dyes: Xanthens (e.g. Fluorescein), Acridines (e.g. Acridine Yellow), Oxazines (e.g. Oxazine 1), Cynines (e.g. Cy7/Cy 3), Styryl dyes (e.g. Dye-28), Coumarines (e.g. Alexa Fluor 350), Porphines (e.g. Chlorophyll B), Metal-Ligand-Complexes (e.g. PtOEPK), Fluorescent proteins (e.g. APC, R-Phycoerythrin), Nanocrystals (e.g. QuantumDot 705), Perylenes (e.g. Lumogen Red F300) and Phtalocyanines (e.g. IRDYE™700DX) as well as conjugates and combinations of these classes of dyes or fluorescent 65 Tb emitting. In some embodiments, the label comprises a fluorescein derivative formed by conjugation of a free amine on the

Throughout this application, various embodiments may be presented in a range formats. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. In some embodiments, the subject may be diagnosed or suspected of being at high risk for cancer. In some embodiments, the subject may be diagnosed or suspected of being at high risk for having a tumor. In some embodiments, the subject is not necessarily diagnosed or suspected of being at high risk for cancer.

A pharmaceutical formulation generally encompasses and/or refers to a preparation which is in such form as to permit the biological activity of an active ingredient contained therein to be effective, and which contains no additional components which are unacceptably toxic to a subject to which the formulation would be administered.

A pharmaceutically acceptable carrier generally encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative.

### Peptides and Peptidomimetics

In one aspect, the present disclosure provides an oligopeptide or peptidomimetic of Formula (Ia) or Formula (Ib):

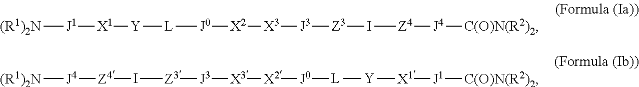

or pharmaceutically acceptable salt thereof, wherein:
J⁰ is a linker region consisting of two to five amino acid residues;
J¹ and J⁴ are each optional linker regions consisting of one to six amino acid residues;
J³ is an optional linker region consisting of one to three amino acid residues;
Z³, Z⁴, Z^{3′}, and Z^{4′} are each an amino acid residue selected from histidine (H), tyrosine (Y), phenylalanine (F), and tryptophan (W);
X¹ and X^{1′} are each a tripeptide region consisting of three amino acid residues, wherein at least one amino acid residue is selected from histidine (H), lysine (K), arginine (R), alanine (A), cysteine (C), and methionine (M), and one amino acid residue is selected from leucine (L), isoleucine (I), methionine (M), and valine (V);
X² and X^{2′} are each independently selected from (i) and (ii):
   (i) when Z³ and Z⁴ are each tyrosine (Y) or when Z^{3′} and Z^{4′} are each tyrosine (Y), X² and X^{2′} are each selected from leucine (L), isoleucine (I), alloisoleucine, methionine (M), serine (S), and threonine (T); or
   (ii) when at least one of Z³ and Z⁴ is not tyrosine (Y) or when at least one of Z^{3′} and Z^{4′} is not tyrosine (Y),
      X² and X^{2′} are each selected from glycine (G), alanine (A), valine (V), leucine (L), isoleucine (I), alloisoleucine, methionine (M), serine (S), threonine (T);
X³ and X^{3′} are each an amino acid residue selected from asparagine (N), glutamine (Q), serine (S), and threonine (T);
R¹ is selected at each occurrence from hydrogen, —C(O)C₁₋₄ alkyl, and B—R³;
R² is selected at each occurrence from hydrogen and B—R³;
   wherein B is absent or an organic linker, and R³ is a label or a water-soluble oligomer; wherein the amino acid residues of X¹, X², X³, Z³ and Z⁴ are L-amino acid residues, and the amino acid residues of X^{1′}, X^{2′}, X^{3′}, Z^{3′} and Z^{4′} are D-amino acid residues.

In some embodiments of the oligopeptide or peptidomimetic of Formula (Ia) or (Ib), J⁰ is a linker region consisting of two to five amino acid residues. In some embodiments, J⁰ is a linker region consisting of two to four amino acid residues. In some embodiments, J⁰ is a linker region consisting of two to three amino acid residues. In some embodiments, J⁰ is a linker region consisting of two amino acid residues. In some embodiment, J⁰ comprises at least one unnatural amino acid residue.

In some embodiments of the oligopeptide or peptidomimetic of Formula (Ia) or (Ib), the oligopeptide or peptidomimetic is represented by Formula (Ia-1) or Formula (Ib-1):

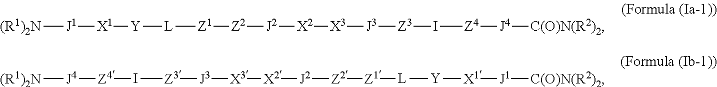

or a pharmaceutically acceptable salt thereof, wherein:
J² and J³ are each an optional linker region consisting of one to three amino acid residues, wherein J² and J³ comprise the same number of amino acid residues; and
Z¹, Z^{1′}, Z², and Z^{2′} are each independently selected from glycine (G), alanine (A), and serine (S), wherein the amino acid residues of Z¹ and Z² are L-amino acid residues, and the amino acid residues of Z^{1′} and Z^{2″} are D-amino acid residues.

In some embodiments of the oligopeptide or peptidomimetic of Formula (Ia), (Ib), (Ia-1), or (Ib-1), Z¹, Z^{1′}, Z², and Z^{2′} are each independently selected from glycine (G) and alanine (A).

In some embodiments of the oligopeptide or peptidomimetic of Formula (Ia), (Ib), (Ia-1), or (Ib-1), Z¹ and Z^{1′} are each independently selected from glycine (G), alanine (A), and serine (S). In some embodiments, Z¹ and Z^{1′} are each independently selected from glycine (G) and alanine (A). In some embodiments, Z¹ and Z^{1′} are each independently selected from glycine (G) and serine (S). In some embodiments, Z¹ and Z^{1′} are each independently selected from alanine (A) and serine (S). In some embodiments, Z¹, and Z^{1′} are each glycine (G). In some embodiments, Z¹ and Z^{1′} are each alanine (A). In some embodiments, Z¹ and Z^{1′} are each serine (S).

In some embodiments of the oligopeptide or peptidomimetic of Formula (Ia), (Ib), (Ia-1), or (Ib-1), Z² and Z^{2′} are each independently selected from glycine (G), alanine (A), and serine (S). In some embodiments, Z² and Z^{2′} are each independently selected from glycine (G) and alanine (A). In some embodiments, Z² and Z^{2′} are each independently selected from glycine (G) and serine (S). In some embodiments Z² and Z^{2′} are each independently selected from alanine (A) and serine (S). In some embodiments, Z² and Z^{2′} are each glycine (G). In some embodiments, Z² and Z^{2′} are each alanine (A).

In some embodiments Z² and Z^{2′} are each serine (S).

In some embodiments of the oligopeptide or peptidomimetic of Formula (Ia), (Ib), (Ia-1), or (Ib-1), at least one of Z¹, Z^{1′}, Z², and Z^{2′} is an unnatural amino acid residue. In some embodiments, at least one of Z¹, Z^{1′}, Z², and Z^{2′} is an unnatural amino acid residue comprising a peptide bond isostere.

In some embodiments, the oligopeptide or peptidomimetic is represented by Formula (Ia) or a pharmaceutically acceptable salt thereof. In some embodiments, the oligopeptide or peptidomimetic is represented by Formula (Ib) or a pharmaceutically acceptable salt thereof. In some embodiments, the oligopeptide or peptidomimetic is represented by Formula (Ia-1) or a pharmaceutically acceptable salt thereof. In some embodiments, the oligopeptide or peptidomimetic is represented by Formula (Ib-1) or a pharmaceutically acceptable salt thereof.

In some embodiments of the oligopeptide or peptidomimetic of Formula (Ia), (Ib), (Ia-1), or (Ib-1), the oligopeptide or peptidomimetic is represented by Formula (Ia-2) or Formula (Ib-2):

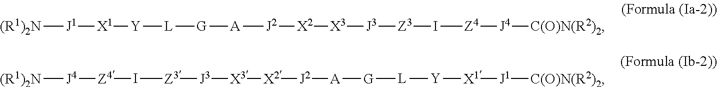

or a pharmaceutically acceptable salt thereof.

In some embodiments of the oligopeptide or peptidomimetic of Formula (Ia), (Ib), (Ia-1), (Ib-1), (Ia-2), or (Ib-2), Z³, Z^{3′}, Z⁴, and Z^{4′} are each independently an amino acid residue selected from histidine (H), tyrosine (Y), and phenylalanine (F). In some embodiments, Z³, Z^{3′}, Z⁴, and Z^{4′} are each independently an amino acid residue selected from tyrosine (Y), phenylalanine (F), and tryptophan (W). In some embodiments, Z³, Z^{3′}, Z⁴, and Z^{4′} are each independently an amino acid residue selected from tyrosine (Y) and phenylalanine (F). In some embodiments, Z³, Z^{3′}, Z⁴, and Z^{4′} are each tyrosine (Y). In some embodiments, Z³, Z^{3′}, Z⁴, and Z^{4′} are each phenylalanine (F).

In some embodiments of the oligopeptide or peptidomimetic of Formula (Ia), (Ib), (Ia-1), (Ib-1), (Ia-2), or (Ib-2), Z³ and Z^{3′} are each an amino acid residue selected from histidine (H), tyrosine (Y), phenylalanine (F), and tryptophan (W). In some embodiments, Z³ and Z^{3′} are each an amino acid residue selected from histidine (H), tyrosine (Y), and phenylalanine (F). In some embodiments, Z³ and Z^{3′} are each an amino acid residue selected from tyrosine (Y), phenylalanine (F), and tryptophan (W). In some embodiments, Z³ and Z^{3′} are each an amino acid residue selected from tyrosine (Y) and phenylalanine (F). In some embodiments, Z³ and Z^{3′} are each tyrosine (Y). In some embodiments, Z³ and Z^{3′} are each phenylalanine (F).

In some embodiments of the oligopeptide or peptidomimetic of Formula (Ia), (Ib), (Ia-1), (Ib-1), (Ia-2), or (Ib-2), Z⁴ and Z^{4′} are each an amino acid residue selected from histidine (H), tyrosine (Y), phenylalanine (F), and tryptophan (W). In some embodiments, Z⁴ and Z^{4′} are each an amino acid residue selected from histidine (H), tyrosine (Y), and phenylalanine (F). In some embodiments, Z⁴ and Z^{4′} are each an amino acid residue selected from tyrosine (Y), phenylalanine (F), and tryptophan (W). In some embodiments, Z⁴ and Z^{4′} are each an amino acid residue selected from tyrosine (Y) and phenylalanine (F). In some embodiments, Z⁴ and Z^{4′} are each tyrosine (Y). In some embodiments, Z⁴ and Z^{4′} are each phenylalanine (F).

In some embodiments of the oligopeptide or peptidomimetic of Formula (Ia), (Ib), (Ia-1), (Ib-1), (Ia-2), or (Ib-2), at least one of Z³, Z^{3′}, Z⁴, and Z^{4′} is an unnatural amino acid residue. In some embodiments, at least one of Z³, Z^{3′}, Z⁴, and Z^{4′} is an unnatural amino acid residue comprising a peptide bond isostere.

In some embodiments, the oligopeptide or peptidomimetic is represented by Formula (Ia-2) or a pharmaceutically acceptable salt thereof. In some embodiments, the oligopeptide or peptidomimetic is represented by Formula (Ib-2) or a pharmaceutically acceptable salt thereof.

In some embodiments of the oligopeptide or peptidomimetic of Formula (Ia) or (Ib), the oligopeptide or peptidomimetic is represented by Formula (Ia-3) or Formula (Ib-3):

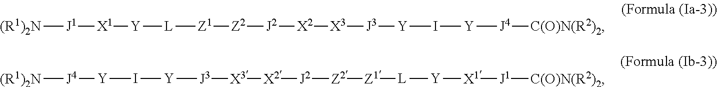

or pharmaceutically acceptable salt thereof.

In some embodiments of the oligopeptide or peptidomimetic of Formula (Ia), (Ib), (Ia-1), (Ib-1), (Ia-2), (Ib-2), (Ia-3), or (Ib-3), Z¹, Z^{1′}, Z², and Z^{2′} are each independently selected from glycine (G) and alanine (A).

In some embodiments of the oligopeptide or peptidomimetic of Formula (Ia), (Ib), (Ia-1), (Ib-1), (Ia-2), (Ib-2), (Ia-3), or (Ib-3), Z¹ and Z^{1′} are each independently selected from glycine (G), alanine (A), and serine (S). In some embodiments, Z¹ and Z^{1′} are each independently selected from glycine (G) and alanine (A). In some embodiments, Z¹, and Z^{1′} are each independently selected from glycine (G) and serine (S). Z¹ and Z^{1′} are each independently selected from alanine (A) and serine (S). In some embodiments, Z¹, and Z^{1′} are each glycine (G). In some embodiments, Z¹, and Z^{1′} are each alanine (A). In some embodiments, Z¹, and Z^{1′} are each serine (S).

In some embodiments of the oligopeptide or peptidomimetic of Formula (Ia), (Ib), (Ia-1), (Ib-1), (Ia-2), (Ib-2), (Ia-3), or (Ib-3), Z² and Z^{2′} are each independently selected from glycine (G), alanine (A), and serine (S). In some embodiments, Z² and Z^{2′} are each independently selected from glycine (G) and alanine (A). In some embodiments, Z² and Z^{2′} are each independently selected from glycine (G) and serine (S). In some embodiments Z² and Z^{2′} are each independently selected from alanine (A) and serine (S).

In some embodiments, Z² and Z^{2′} are each glycine (G). In some embodiments, Z² and Z^{2′} are each alanine (A). In some embodiments Z² and Z^{2′} are each serine (S).

In some embodiments, the oligopeptide or peptidomimetic is represented by Formula (Ia-3) or a pharmaceutically acceptable salt thereof. In some embodiments, the oligopeptide or peptidomimetic is represented by Formula (Ib-3) or a pharmaceutically acceptable salt thereof.

In some embodiments of the oligopeptide or peptidomimetic of Formula (Ia), (Ib), (Ia-1), (Ib-1), (Ia-2), (Ib-2), (Ia-3), or (Ib-3), the oligopeptide or peptidomimetic is represented by Formula Ia-3) or Formula Ib-3):

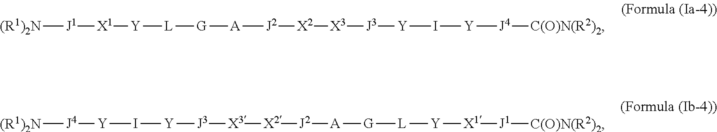

or pharmaceutically acceptable salt thereof.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), X¹ and X^{1′} are each a tripeptide region consisting of three amino acid residues, wherein at least one amino acid residue is selected from histidine (H), lysine (K), arginine (R), and cysteine (C). In some embodiments, X¹ and X^{1′} are each a tripeptide region consisting of three amino acid residues, wherein at least one amino acid residue is selected from histidine (H), lysine (K), and arginine (R). In some embodiments, X¹ and X^{1′} are each a tripeptide region consisting of three amino acid residues, wherein at least one amino acid residue is selected from histidine (H) and lysine (K). In some embodiments, X¹ and X^{1′} are each a tripeptide region consisting of three amino acid residues, wherein at least one amino acid residue is selected from histidine (H) and arginine (R). In some embodiments, X¹ and X^{1′} are each a tripeptide region consisting of three amino acid residues, wherein at least one amino acid residue is selected from histidine (H).

In some embodiments, (Ia), (Ib), (Ia-1), (Ib-1), (Ia-2), (Ib-2), (Ia-3), (Ib-3), (Ia-4) or (Ib-4), X¹ and X^{1′} are each a tripeptide region consisting of three amino acid residues, wherein at least two amino acid residue is selected from histidine (H), lysine (K), and arginine (R). In some embodiments, X¹ and X^{1′} are each a tripeptide region consisting of three amino acid residues, wherein at least two amino acid residue is selected from histidine (H) and lysine (K). In some embodiments, X¹ and X^{1′} are each a tripeptide region consisting of three amino acid residues, wherein at least two amino acid residue is selected from histidine (H) and arginine (R). In some embodiments, X¹ and X^{1′} are each a tripeptide region consisting of three amino acid residues, wherein at least two amino acid residue is selected from histidine (H).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), at least two consecutive amino acid residues of X¹ and X^{1′} are selected from histidine (H), lysine (K), and arginine (R).

In some embodiments, X¹ and X^{1′} are each a tripeptide region consisting of three amino acid residues, wherein at least two consecutive amino acid residue is selected from histidine (H) and lysine (K). In some embodiments, X¹ and X^{1′} are each a tripeptide region consisting of three amino acid residues, wherein at least two consecutive amino acid residue is selected from histidine (H) and arginine (R). In some embodiments, X¹ and X^{1′} are each a tripeptide region consisting of three amino acid residues, wherein at least two consecutive amino acid residue is selected from histidine (H).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), X¹ and X^{1′} are each a tripeptide region consisting of three amino acid residues, wherein one amino acid residue is selected from leucine (L), isoleucine (I), and valine (V). In some embodiments, X¹ and X^{1′} are each a tripeptide region consisting of three amino acid residues, wherein one amino acid residue is selected from isoleucine (I) and valine (V). In some embodiments, X¹ and X^{1′} are each a tripeptide region consisting of three amino acid residues, wherein one amino acid residue is selected from leucine (L) and isoleucine (I). In some embodiments, X¹ and X^{1′} are each a tripeptide region consisting of three amino acid residues, wherein one amino acid residue is isoleucine (I).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), X¹ and X^{1′} consist of the following amino acid sequence: —H—H—I—.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), X² and X^{2′} are each independently selected from (i) and (ii):
(i) when Z³ and Z⁴ are each tyrosine (Y), Z¹ is glycine (G), and Z² is alanine (A) or when Z^{3′} and Z^{4′} are each tyrosine (Y), Z^{1′} is glycine (G), and Z^{2′} is alanine (A);
   X² and X^{2′} are each selected from leucine (L), isoleucine (I), alloisoleucine, methionine (M), serine (S), and threonine (T); or
(ii) when at least one of Z³ and Z⁴ is not tyrosine (Y), Z¹ is not glycine (G), or Z² is not alanine (A), or when at least one of Z³ and Z^{4′} is not tyrosine (Y) or Z^{1′} is not glycine (G), Z^{2′} is not alanine (A),
   X² and X^{2′} are each selected from glycine (G), alanine (A), valine (V), leucine (L), isoleucine (I), alloisoleucine, methionine (M), serine (S), threonine (T).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), and X^{2′} are each independently selected from (i) and (ii):
(i) when Z³ and Z⁴ are each tyrosine (Y), Z¹ is glycine (G), and Z² is alanine (A) or when Z^{3′} and Z^{4′} are each tyrosine (Y), Z^{1′} is glycine (G), and Z^{2′} is alanine (A); X² and X^{2′} are each selected from leucine (L), isoleucine (I), and alloisoleucine; or
(ii) when at least one of Z³ and Z⁴ is not tyrosine (Y), Z¹ is not glycine (G), or Z² is not alanine (A), or when at least one of Z^{3′} and Z^{4′} is not tyrosine (Y) or Z^{1′} is not glycine (G), Z^{2′} is not alanine (A),
   X² and X^{2′} are each selected from glycine (G), alanine (A), valine (V), serine (S), and threonine (T).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), Z³, Z^{3′}, Z⁴ and Z^{4′} are each tyrosine (Y), Z¹ is glycine (G), Z² is alanine (A), Z^{1′} is glycine (G), and Z^{2′} is alanine (A); and X² and X^{2′} are each selected from leucine (L), isoleucine (I), alloisoleucine, methionine (M), serine (S), and threonine (T). In some embodiments, Z³, Z³, Z⁴ and Z⁴ are each tyrosine (Y), Z¹ is glycine (G), Z² is alanine (A), Z^{1′} is glycine (G), and Z^{2′} is alanine (A); and X² and X^{2′} are each selected from leucine (L), isoleucine (I), alloisoleucine, and methionine (M). In some embodiments, Z³, Z^{3′}, Z⁴ and Z^{4′} are each tyrosine (Y), Z¹ is glycine (G), Z² is alanine (A), Z^{1′} is glycine (G), and Z^{2′} is alanine (A); and X² and X^{2′} are each selected from leucine (L), isoleucine (I), and alloisoleucine.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), at least one of Z³ and Z⁴ is not tyrosine (Y), Z¹ is not glycine (G), or Z² is not alanine (A), or when at least one of Z^{3′} and Z^{4′} is not tyrosine (Y) or Z^{1′} is not glycine (G), Z^{2′} is not alanine (A); and X² and X^{2′} are each selected from glycine (G), alanine (A), valine (V), leucine (L), isoleucine (I), alloisoleucine, methionine (M), serine (S), and threonine (T).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), at least one of Z³ and Z⁴ is not tyrosine (Y), Z¹ is not glycine (G), or Z² is not alanine (A), or when at least one of Z^{3′} and Z^{4′} is not tyrosine (Y) or Z^{1′} is not glycine (G), Z^{2′} is not alanine (A); and X² and X^{2′} are each selected from glycine (G), alanine (A), valine (V), leucine (L), isoleucine (I), alloisoleucine, serine (S), and threonine (T). In some embodiments, In some embodiments, at least one of Z³ and Z⁴ is not tyrosine (Y), Z¹ is not glycine (G), or Z² is not alanine (A), or when at least one of Z^{3′} and Z^{4′} is not tyrosine (Y) or Z^{1′} is not glycine (G), Z^{2′} is not alanine (A); and X² and X^{2′} are each selected from glycine (G), alanine (A), valine (V), serine (S), and threonine (T). In some embodiments, X² and X^{2′} are each an amino acid residue selected from glycine (G), alanine (A), valine (V), serine (S), and threonine (T). In some embodiments, X² and X^{2′} are each an amino acid residue selected from glycine (G), alanine (A), serine (S), and threonine (T). In some embodiments, X² and X^{2′} are each glycine (G). In some embodiments, X² and X^{2′} are each alanine (A). In some embodiments, X² and X^{2′} are each valine (V). In some embodiments, X² and X^{2′} are each serine (S). In some embodiments, X² and X^{2′} are each threonine (T).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), X³ and X^{3′} are each an amino acid residue selected from asparagine (N), glutamine (Q), serine (S), and threonine (T).

In some embodiments, X³ and X^{3′} are each an amino acid residue selected from asparagine (N), serine (S), and threonine (T). In some embodiments, X³ and X^{3′} are each an amino acid residue selected from asparagine (N), glutamine (Q), and threonine (T). In some embodiments, X³ and X^{3′} are each an amino acid residue selected from asparagine (N), glutamine (Q), and serine (S). In some embodiments, X³ and X^{3′} are each an amino acid residue selected from asparagine (N) and serine (S). In some embodiments, X³ and X^{3′} are each an amino acid residue selected from asparagine (N) and threonine (T). In some embodiments, X³ and X^{3′} are each an amino acid residue selected from asparagine (N) and glutamine (Q). In some embodiments, X³ and X^{3′} are each asparagine (N).

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), J¹, J², J³, and J⁴ are each absent. In some embodiments, J¹, J², and J³ are each absent. In some embodiments, J², J³, and J⁴ are each absent. In some embodiments, J¹, J², and J⁴ are each absent. In some embodiments, J¹, J³, and J⁴ are each absent. In some embodiments, J¹ and J² are each absent. In some embodiments, J¹ and J³ are each absent. J¹ and J⁴ are each absent. In some embodiments, J² and J³ are each absent. In some embodiments, J² and J⁴ are each absent. In some embodiments, J³ and J⁴ are each absent. In some embodiments, J¹ is absent. In some embodiments, J² is absent. In some embodiments, J³ is absent. In some embodiments, J⁴ is absent.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), J¹, J², and J³ are each optional linker regions consisting of one to five amino acid residues. In some embodiments, J¹, J², J³, and J⁴ are each optional linker regions consisting of one to four amino acid residues. In some embodiments, J¹, J², and J³ are each optional linker regions consisting of one to three amino acid residues. In some embodiments, J¹, J², J³, and J⁴ are each optional linker regions consisting of one to two amino acid residues. In some embodiments, J¹, J², J³, and J⁴ are each optional linker regions consisting of one amino acid residue.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), J² and J³ comprise the same number of amino acid residues. In some embodiments, J² and J³ comprise a different number of amino acid residues. In some embodiment, J² and J³ each consist of one to three amino acid residues. In some embodiment, J² and J³ each consist of one to two amino acid residues. In some embodiment, J² and J³ each consist of two to three amino acid residues. In some embodiment, J² and J³ each consist of one amino acid residue. In some embodiment, J² and J³ each consist of two amino acid residues. In some embodiment, J² and J³ each consist of three amino acid residues.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), at least one of J¹, J², J³, and J⁴ is a peptide linker region consisting of one to six amino acid residues. In some embodiments, at least one of J¹, J², J³, and J⁴ is a peptide linker region consisting of one to five amino acid residues. In some embodiments, at least one of J¹, J², J³, and J⁴ is a peptide linker region consisting of one to four amino acid residues. In some embodiments, at least one of J¹, J², J³, and J⁴ is a peptide linker region consisting of one to three amino acid residues. In some embodiments, at least one of J¹, J², J³, and J⁴ is a peptide linker region consisting of one to two amino acid residues.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), at least one of J¹, J², and J³ is a peptide linker region consisting of at least one unnatural amino acid residue. In some embodiments, at least one of J¹, J², J³, and J⁴ is a peptide linker region consisting of at least two unnatural amino acid residue. In some embodiments, at least three of J¹, J², J³, and J⁴ is a peptide linker region consisting of at least two unnatural amino acid residue.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), R¹ is selected at each occurrence from hydrogen and B—R³. In some embodiments, at least one R¹ is hydrogen. In some embodiments, each occurrence of R¹ is hydrogen.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), is selected at each occurrence from hydrogen and B—R³. In some embodiments, at least one R² is hydrogen. In some embodiments, each occurrence of R² is hydrogen. In some embodiments, each occurrence of R¹ and R² is hydrogen. In some embodiments, at least one occurrence of R¹ and R² is B—R³.

In some embodiments, B is absent or an organic linker, and R³ is a label or a water-soluble oligomer. In some embodiments, B is absent. In some embodiments, B is an organic linker. In some embodiments, B is an organic linker comprising polyethylene glycol (PEG). In some embodiments, R³ is a label. In some embodiments, R³ is a fluorescent label. In some embodiments, R³ is a label. In some embodiments, R³ is a water-soluble oligomer. In some embodiments, R³ is a water-soluble oligomer comprising PEG.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

In some embodiments of the oligopeptide or peptidomimetic of Formula (Ia), (Ib), (Ia-1), (Ib-1), (Ia-2), (Ib-2), (Ia-3), (Ib-3), (Ia-4) or (Ib-4), at least one amino acid residue of Z¹, Z^{1′}, Z², Z^{2′}, Z³, Z^{3′}, Z⁴, and Z^{4′} is an unnatural amino acid residue. In some embodiments, at least one amino acid residue of Z³, Z^{3′}, Z⁴, and Z^{4′} is an unnatural amino acid residue having a side chain comprising a substituted phenyl ring.

In some embodiments, the oligopeptide or peptidomimetic is represented by Formula (Ia-4) or a pharmaceutically acceptable salt thereof. In some embodiments, the oligopeptide or peptidomimetic is represented by Formula (Ib-4) or a pharmaceutically acceptable salt thereof.

In another aspect, the present disclosure provides an oligopeptide or peptidomimetic of Formula (IIa) or Formula (IIb):

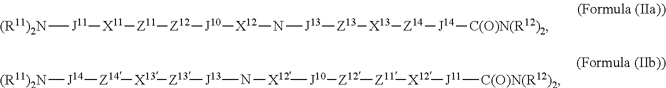

or pharmaceutically acceptable salt thereof, wherein:
J¹⁰ is a linker region consisting of two to five amino acid residues;
J¹¹ and J¹⁴ and are each optional linker regions consisting of one to six amino acid residues;
J¹³ is an optional linker region consisting of one to three amino acid residues;
Z¹¹ and Z^{11′} are each an amino acid residue selected from: tyrosine (Y), phenylalanine (F), tryptophan (W), alanine (A), valine, (V), leucine (L), isoleucine (I), alloisoleucine, and methionine (M);
Z¹² and Z^{12′} are each an amino acid residue selected from: glycine (G), alanine (A), valine (V), leucine (L), isoleucine (I), and alloisoleucine;
Z¹³, Z¹⁴, Z^{13′}, and Z^{14′} are each an amino acid residue selected from histidine (H), tyrosine (Y) phenylalanine (F), and tryptophan (W);
X¹¹ and X^{11′} are each a tripeptide region consisting of three amino acid residues, wherein at least one amino acid residue is selected from histidine (H), lysine (K), arginine (R), alanine (A), cysteine (C), and methionine (M), and one amino acid residue is selected from leucine (L), isoleucine (I), methionine (M), and valine (V); provided that at most one of the three amino acid residues of X¹¹ and X^{11′} is histidine;
X¹² and X^{12′} are each an amino acid residue selected from glycine (G), alanine (A), valine (V), leucine (L), isoleucine (I), and methionine (M);
X¹³ and X^{13′} are each an amino acid residue selected from valine (V), leucine (L), isoleucine (I), alloisoleucine, methionine (M), phenylalanine (F), and tyrosine (Y);
R¹¹ is selected at each occurrence from hydrogen, —C(O)C₁₋₄ alkyl, and B—R¹³;
R¹² is selected at each occurrence from hydrogen and B—R¹³;
   wherein B is absent or an organic linker, and R¹³ is a label or a water-soluble oligomer;
wherein the amino acids of X¹¹, X¹², Z¹¹, Z¹², Z¹³, and Z¹⁴ are L-amino acids, and the amino acids of X^{11′}, X^{12′}, Z^{11′}, Z^{12′}, Z^{13′}, and Z^{14′} are D-amino acids.

In some embodiments of the oligopeptide or peptidomimetic of Formula (IIa) or (IIb), J¹⁰ is a linker region consisting of two to five amino acid residues. In some embodiments, J¹⁰ is a linker region consisting of two to four amino acid residues. In some embodiments, J¹⁰ is a linker region consisting of two to three amino acid residues. In some embodiments, J¹⁰ is a linker region consisting of two amino acid residues. In some embodiment, J¹⁰ comprises at least one unnatural amino acid residue.

In some embodiments of the oligopeptide or peptidomimetic of Formula (IIa) or (IIb), the oligopeptide or peptidomimetic is represented by Formula (IIa-1) or Formula (IIb-1):

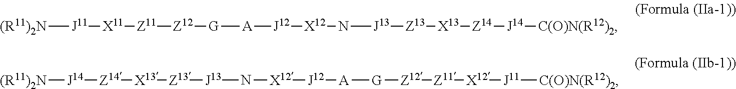

or a pharmaceutically acceptable salt thereof, wherein:
J¹² and J¹³ are each an optional linker region consisting of one to three amino acid residues, wherein J¹² and J¹³ comprise the same number of amino acid residues.

In some embodiments of the oligopeptide or peptidomimetic of Formula (IIa), (IIb), (IIa-1), or (IIb-1), Z¹¹ and Z^{11′} are each an amino acid residue selected from: tyrosine (Y), phenylalanine (F), tryptophan (W), alanine (A), valine, (V), leucine (L), isoleucine (I), alloisoleucine, and methionine (M). In some embodiments, Z¹¹ and Z^{11′} are each an amino acid residue selected from: tyrosine (Y), phenylalanine (F), tryptophan (W), valine, (V), leucine (L), isoleucine (I), alloisoleucine, and methionine (M). In some embodiments, Z¹¹ and Z^{11′} are each an amino acid residue selected from: tyrosine (Y), phenylalanine (F), tryptophan (W), alanine (A), valine, (V), leucine (L), and isoleucine (I). In some embodiments, Z¹¹ and Z^{11′} are each an amino acid residue selected from: tyrosine (Y), phenylalanine (F), tryptophan (W), valine, (V), leucine (L), and isoleucine (I). In some embodiments, Z¹¹ and Z^{11′} are each an amino acid residue selected from: tyrosine (Y), phenylalanine (F), tryptophan (W), and alanine (A). In some embodiments, Z¹¹ and Z^{11′} are each an amino acid residue selected from: tyrosine (Y), phenylalanine (F), and tryptophan (W). In some embodiments, Z¹¹ and Z^{11′} are each an amino acid residue selected from: tyrosine (Y) and phenylalanine (F). In some embodiments, Z¹¹ and Z^{11′} are each an amino acid residue selected from: tyrosine (Y) and tryptophan (W). In some embodiments, Z¹¹ and Z^{11′} are each tyrosine (Y).

In some embodiments of the oligopeptide or peptidomimetic of Formula (IIa), (IIb), (IIa-1), or (IIb-1), Z¹² and Z^{12′} are each an amino acid residue selected from: glycine (G), alanine (A), valine (V), leucine (L), and isoleucine (I). In some embodiments, Z¹² and Z^{12′} are each an amino acid residue selected from: glycine (G), alanine (A), valine (V), and leucine (L). In some embodiments, Z¹² and Z^{12′} are each an amino acid residue selected from: glycine (G), alanine (A) and leucine (L). In some embodiments, Z¹² and Z^{12′} are each glycine (G). In some embodiments, Z¹² and Z^{12′} are each alanine (A). In some embodiments, Z¹² and Z^{12′} are each leucine (L).

In some embodiments of the oligopeptide or peptidomimetic of Formula (IIa), (IIb), (IIa-1), or (IIb-1), at least one of Z¹¹, Z^{11′}, Z¹², and Z^{12′} is an unnatural amino acid residue. In some embodiments, at least one of Z¹¹, Z^{11′}, Z¹², and Z^{12′} is an unnatural amino acid residue comprising a peptide bond isostere.

In some embodiments, the oligopeptide or peptidomimetic is represented by Formula (IIa) or a pharmaceutically acceptable salt thereof. In some embodiments, the oligopeptide or peptidomimetic is represented by Formula (IIb) or a pharmaceutically acceptable salt thereof. In some embodiments, the oligopeptide or peptidomimetic is represented by Formula (IIa-1) or a pharmaceutically acceptable salt thereof. In some embodiments, the oligopeptide or peptidomimetic is represented by Formula (IIb-1) or a pharmaceutically acceptable salt thereof.

In some embodiments of the oligopeptide or peptidomimetic of Formula (IIa) or (IIb), the oligopeptide or peptidomimetic is represented by Formula (IIa-2) or Formula (IIb-2):

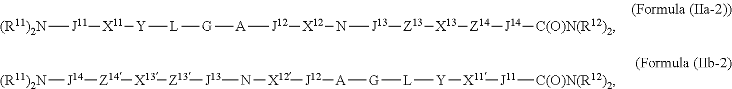

or pharmaceutically acceptable salt thereof.

In some embodiments of the oligopeptide or peptidomimetic of Formula (IIa), (IIb), (IIa-1), (IIb-1), (IIa-2), or (IIb-2), Z¹³, Z¹⁴, Z^{13′}, and Z^{14′} are each phenylalanine (F).

In some embodiments of the oligopeptide or peptidomimetic of Formula (IIa), (IIb), (IIa-1), (IIb-1), (IIa-2), or (IIb-2), Z¹³ and Z^{13′} are each an amino acid residue selected from tyrosine (Y) and phenylalanine (F). In some embodiments, Z¹³ and Z^{13′} are each phenylalanine (F).

In some embodiments of the oligopeptide or peptidomimetic of Formula (IIa), (IIb), (IIa-1), (IIb-1), (IIa-2), or (IIb-2), Z¹⁴ and Z^{14′} are each an amino acid residue selected from tyrosine (Y) and phenylalanine (F). In some embodiments, Z¹⁴ and Z^{14′} are each phenylalanine (F).

In some embodiments of the oligopeptide or peptidomimetic of Formula (IIa), (IIb), (IIa-1), (IIb-1), (IIa-2), or (IIb-2), X¹³ and X^{13′} are each an amino acid residue selected from leucine (L), isoleucine (I), and alloisoleucine. In some embodiments, X¹³ and X^{13′} are each an amino acid residue selected from isoleucine (I) and alloisoleucine.

In some embodiments, X¹³ and X^{13′} are each an amino acid residue selected from leucine (L) and isoleucine (I). In some embodiments, X¹³ and X^{13′} are each isoleucine (I).

In some embodiments of the oligopeptide or peptidomimetic of Formula (IIa), (IIb), (IIa-1), (IIb-1), (IIa-2), or (IIb-2), at least one of Z¹³, Z^{13′}, Z¹⁴, and Z^{14′} is an unnatural amino acid residue. In some embodiments, at least one Z¹³, Z^{13′}, Z¹⁴, and Z^{14′} is an unnatural amino acid residue comprising a peptide bond isostere. In some embodiments, at least one of Z¹³, Z^{13′}, Z¹⁴, and Z^{14′} is an unnatural amino acid residue having a side chain comprising a substituted phenyl group. In some embodiments, X¹³ and X^{13′} are each an unnatural amino acid residue. In some embodiments, X¹³ and X^{13′} are each an unnatural amino acid residue comprising a peptide bond isostere.

In some embodiments, the oligopeptide or peptidomimetic is represented by Formula (IIa-2) or a pharmaceutically acceptable salt thereof. In some embodiments, the oligopeptide or peptidomimetic is represented by Formula (IIb-2) or a pharmaceutically acceptable salt thereof.

In some embodiments of the oligopeptide or peptidomimetic of Formula (IIa), (IIb), (IIa-1), (IIb-1), (IIa-2), or (IIb-2), the oligopeptide or peptidomimetic is represented by Formula (IIa-3) or Formula (IIb-3):

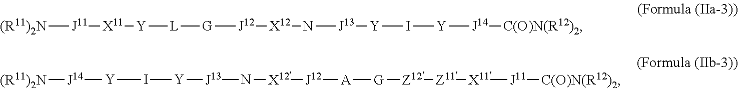

or pharmaceutically acceptable salt thereof.

In some embodiments of the oligopeptide or peptidomimetic of Formula (IIa), (IIb), (IIa-1), (IIb-1), (IIa-2), (IIb-2), (IIa-3), or (IIb-3), Z¹¹ and Z^{11′} are each an amino acid residue selected from: tyrosine (Y), phenylalanine (F), tryptophan (W), alanine (A), valine, (V), leucine (L), isoleucine (I), alloisoleucine, and methionine (M). In some embodiments, Z¹¹ and Z^{11′} are each an amino acid residue selected from: tyrosine (Y), phenylalanine (F), tryptophan (W), valine, (V), leucine (L), isoleucine (I), alloisoleucine, and methionine (M). In some embodiments, Z¹¹ and Z^{11′} are each an amino acid residue selected from: tyrosine (Y), phenylalanine (F), tryptophan (W), alanine (A), valine, (V), leucine (L), and isoleucine (I). In some embodiments, Z¹¹ and Z^{11′} are each an amino acid residue selected from: tyrosine (Y), phenylalanine (F), tryptophan (W), valine, (V), leucine (L), and isoleucine (I). In some embodiments, Z¹¹ and Z^{11′} are each an amino acid residue selected from: tyrosine (Y), phenylalanine (F), tryptophan (W), and alanine (A).

In some embodiments, Z¹¹ and Z^{11′} are each an amino acid residue selected from: tyrosine (Y), phenylalanine (F), and tryptophan (W). In some embodiments, Z¹¹ and Z^{11′} are each an amino acid residue selected from: tyrosine (Y) and phenylalanine (F). In some embodiments, Z¹¹ and Z^{11′} are each an amino acid residue selected from: tyrosine (Y) and tryptophan (W). In some embodiments, Z¹¹ and Z^{11′} are each tyrosine (Y).

In some embodiments of the oligopeptide or peptidomimetic of Formula (IIa), (IIb), (IIa-1), (IIb-1), (IIa-2), (IIb-2), (IIa-3), or (IIb-3), Z¹² and Z^{12′} are each an amino acid residue selected from: glycine (G), alanine (A), valine (V), leucine (L), and isoleucine (I). In some embodiments, Z¹² and Z^{12′} are each an amino acid residue selected from: glycine (G), alanine (A), valine (V), and leucine (L). In some embodiments, Z¹² and Z^{12′} are each an amino acid residue selected from: glycine (G), alanine (A) and leucine (L). In some embodiments, Z¹² and Z^{12′} are each glycine (G). In some embodiments, Z¹² and Z^{12′} are each alanine (A). In some embodiments, Z¹² and Z^{12′} are each leucine (L).

In some embodiments of the oligopeptide or peptidomimetic of Formula (IIa), (IIb), (IIa-1), (IIb-1), (IIa-2), (IIb-2), (IIa-3), or (IIb-3), at least one of Z¹¹, Z^{11′}, Z¹², and Z^{12′} is an unnatural amino acid residue. In some embodiments, at least one Z¹¹, Z^{11′}, Z¹², and Z^{12′} is an unnatural amino acid residue comprising a peptide bond isostere.

In some embodiments, the oligopeptide or peptidomimetic is represented by Formula (IIa-3) or a pharmaceutically acceptable salt thereof. In some embodiments, the oligopeptide or peptidomimetic is represented by Formula (IIb-3) or a pharmaceutically acceptable salt thereof.

In some embodiments of the oligopeptide or peptidomimetic of Formula (IIa), (IIb), (IIa-1), (IIb-1), (IIa-2), (IIb-2), (IIa-3), or (IIb-3), the oligopeptide or peptidomimetic is represented by Formula (IIa-4) or Formula (IIb-4):

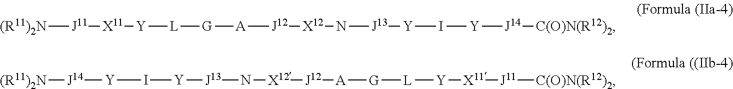

or pharmaceutically acceptable salt thereof.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), X¹¹ and X^{11′} are each a tripeptide region consisting of three amino acid residues, wherein at least one amino acid residue is selected from histidine (H), lysine (K), arginine (R), and cysteine (C), provided that at most one of the three amino acid residues of X¹¹ and X^{11′} is histidine.

In some embodiments, X¹¹ and X^{11′} are each a tripeptide region consisting of three amino acid residues, wherein at least one amino acid residue is selected from histidine (H), lysine (K), and arginine (R). In some embodiments, X¹¹ and X^{11′} are each a tripeptide region consisting of three amino acid residues, wherein at least one amino acid residue is selected from histidine (H) and lysine (K). In some embodiments, X¹¹ and X^{11′} are each a tripeptide region consisting of three amino acid residues, wherein at least one amino acid residue is selected from histidine (H) and arginine (R). In some embodiments, X¹¹ and X^{11′} are each a tripeptide region consisting of three amino acid residues, wherein one amino acid residue is selected from histidine (H).

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), X¹¹ and X^{11′} are each a tripeptide region consisting of three amino acid residues, wherein at least two amino acid residue is selected from histidine (H), lysine (K), and arginine (R). In some embodiments, X¹¹ and X^{11′} are each a tripeptide region consisting of three amino acid residues, wherein at least two amino acid residue is selected from histidine (H) and lysine (K). In some embodiments, X¹¹ and X^{11′} are each a tripeptide region consisting of three amino acid residues, wherein at least two amino acid residue is selected from histidine (H) and arginine (R). In some embodiments, X¹¹ and X^{11′} are each a tripeptide region consisting of three amino acid residues, wherein at least two amino acid residue is selected from histidine (H).

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), X¹¹ and X^{11′} are each a tripeptide region consisting of three amino acid residues, wherein one amino acid residue is selected from leucine (L), isoleucine (I), and valine (V). In some embodiments, X¹¹ and X^{11′} are each a tripeptide region consisting of three amino acid residues, wherein one amino acid residue is selected from isoleucine (I) and valine (V).

In some embodiments, X¹¹ and X^{11′} are each a tripeptide region consisting of three amino acid residues, wherein one amino acid residue is selected from leucine (L) and isoleucine (I). In some embodiments, X¹¹ and X^{11′} are each a tripeptide region consisting of three amino acid residues, wherein one amino acid residue is isoleucine (I).

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), X¹² and X^{12′} are each an amino acid residue selected from glycine (G), alanine (A), valine (V), leucine (L), isoleucine (I), and methionine (M). In some embodiments, X¹² and X^{12′} are each an amino acid residue selected from alanine (A), valine (V), leucine (L), isoleucine (I), and methionine (M). In some embodiments, X¹² and X^{12′} are each an amino acid residue selected from valine (V), leucine (L), isoleucine (I), and methionine (M). In some embodiments, X¹² and X^{12′} are each an amino acid residue selected from alanine (A), valine (V), leucine (L), and isoleucine (I). In some embodiments, X¹² and X^{12′} are each an amino acid residue selected from valine (V), leucine (L), and isoleucine (I). In some embodiments, X¹² and X^{12′} are each an amino acid residue selected from valine (V) and isoleucine (I). In some embodiments, X¹² and X^{12′} are each an amino acid residue selected from valine (V) and leucine (L). In some embodiments, X¹² and X^{12′} are each valine (V).

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), X¹³ and X^{13′} are each an amino acid residue selected from asparagine (N), glutamine (Q), serine (S), and threonine (T). In some embodiments, X¹³ and X^{13′} are each an amino acid residue selected from asparagine (N), serine (S), and threonine (T). In some embodiments, X¹³ and X^{13′} are each an amino acid residue selected from asparagine (N), glutamine (Q), and threonine (T). In some embodiments, X¹³ and X^{13′} are each an amino acid residue selected from asparagine (N), glutamine (Q), and serine (S). In some embodiments, X¹³ and X^{13′} are each an amino acid residue selected from asparagine (N) and serine (S). In some embodiments, X¹³ and X^{13′} are each an amino acid residue selected from asparagine (N) and threonine (T). In some embodiments, X¹³ and X^{13′} are each an amino acid residue selected from asparagine (N) and glutamine (Q). In some embodiments, X¹³ and X^{13′} are each asparagine (N).

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), at least one amino acid residue is an unnatural amino acid residue. In some embodiments, at least one amino acid residue is an unnatural amino acid residue comprising a peptide bond isostere.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), J¹¹, J¹², J¹³, and J¹⁴ are each optional linker regions consisting of one to five amino acid residues. In some embodiments, J¹¹, J¹², J¹³, and J¹⁴ are each optional linker regions consisting of one to four amino acid residues. In some embodiments, J¹¹, J¹², J¹³, and J¹⁴ are each optional linker regions consisting of one to three amino acid residues. In some embodiments, J¹¹, J¹², J¹³, and J¹⁴ are each optional linker regions consisting of one to two amino acid residues. In some embodiments, J¹¹, J¹², J¹³, and J¹⁴ are each optional linker regions consisting of one amino acid residue.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), J¹¹, J¹², J¹³, and J¹⁴ are each absent. In some embodiments, J¹¹, J¹², and J¹³ are each absent. In some embodiments, J¹², J¹³, and J¹⁴ are each absent. In some embodiments, J¹¹, J¹², and J¹⁴ are each absent. In some embodiments, J¹¹, J¹³, and J¹⁴ are each absent. In some embodiments, J¹¹ and J¹² are each absent. In some embodiments, J¹¹ and J¹³ are each absent. J¹¹ and J¹⁴ are each absent. In some embodiments, J¹² and J¹³ are each absent. In some embodiments, J¹² and J¹⁴ are each absent. In some embodiments, J¹³ and J¹⁴ are each absent. In some embodiments, J¹¹ is absent. In some embodiments, J¹² is absent. In some embodiments, J¹³ is absent. In some embodiments, J¹⁴ is absent.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), at least one of J¹¹, J¹², J¹³, and J¹⁴ is a peptide linker region consisting of one to six amino acid residues.

In some embodiments, at least one of J¹¹, J¹², J¹³, and J¹⁴ is a peptide linker region consisting of one to five amino acid residues. In some embodiments, at least one of J¹¹, J¹², J¹³, and J¹⁴ is a peptide linker region consisting of one to four amino acid residues.

In some embodiments, at least one of J¹¹, J¹², J¹³ and J¹⁴ is a peptide linker region consisting of one to three amino acid residues. In some embodiments, at least one of J¹¹, J¹², J¹³, and J¹⁴ is a peptide linker region consisting of one to two amino acid residues.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), J¹² and J¹³ comprise the same number of amino acid residues. In some embodiments, J¹² and J¹³ comprise a different number of amino acid residues. In some embodiment, J¹² and J¹³ each consist of one to three amino acid residues. In some embodiment, J¹² and J¹³ each consist of one to two amino acid residues. In some embodiment, J¹² and J¹³ each consist of two to three amino acid residues. In some embodiment, J¹² and J¹³ each consist of one amino acid residue. In some embodiment, J¹² and J¹³ each consist of two amino acid residues. In some embodiment, J¹² and J¹³ each consist of three amino acid residues.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), at least one of J¹¹, J¹², J¹³, and J¹⁴ is a peptide linker region comprising at least one unnatural amino acid residue. In some embodiments, at least one of J¹¹, J¹², J¹³, and J¹⁴ is a peptide linker region comprising at least two unnatural amino acid residue. In some embodiments, at least three of J¹¹, J¹², J¹³, and J¹⁴ is a peptide linker region comprising at least two unnatural amino acid residue.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), R¹¹ is selected at each occurrence from hydrogen and B—R¹³. In some embodiments, at least one R¹¹ is hydrogen. In some embodiments, each occurrence of R¹¹ is hydrogen.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), R¹² is selected at each occurrence from hydrogen and B—R¹³. In some embodiments, at least one R¹² is hydrogen. In some embodiments, each occurrence of R¹² is hydrogen. In some embodiments, each occurrence of R¹¹ and R¹² is hydrogen. In some embodiments, at least one occurrence of R¹¹ and R¹² is B—R¹³.

In some embodiments, B is absent or an organic linker, and R¹³ is a label or a water-soluble oligomer. In some embodiments, B is absent. In some embodiments, B is an organic linker. In some embodiments, B is an organic linker comprising polyethylene glycol (PEG). In some embodiments, R¹³ is a label. In some embodiments, R¹³ is a fluorescent label. In some embodiments, R¹³ is a label. In some embodiments, R¹³ is a water-soluble oligomer. In some embodiments, R¹³ is a water-soluble oligomer comprising PEG.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

In some embodiments of the oligopeptide or peptidomimetic of Formula (IIa), (IIb), (IIa-1), (IIb-1), (IIa-2), (IIb-2), (IIa-3), (IIb-3), (IIa-4) or (IIb-4), at least one amino acid residue of Z¹¹, Z^{11′}, Z¹², Z^{12′}, Z¹³, Z^{13′}, Z¹⁴, and Z^{14′} is an unnatural amino acid residue. In some embodiments, at least one amino acid residue of Z¹³, Z¹³, Z¹⁴, and Z^{14′} is an unnatural amino acid residue having a side chain comprising a substituted phenyl ring.

In some embodiments, the oligopeptide or peptidomimetic is represented by Formula (IIa-4) or a pharmaceutically acceptable salt thereof. In some embodiments, the oligopeptide or peptidomimetic is represented by Formula (IIb-4) or a pharmaceutically acceptable salt thereof.

In another aspect, the present disclosure provides an oligopeptide or peptidomimetic of Formula (IIIa) or Formula (IIIb):

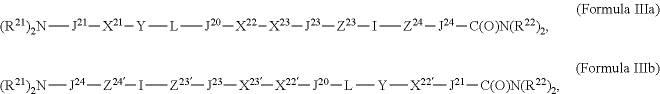

or pharmaceutically acceptable salt thereof, wherein
J²⁰ is a linker region consisting of two to five amino acid residues;
J²¹ and J²⁴ are each optional linker regions consisting of one to six amino acid residues;
J²³ is an optional linker region consisting of one to three amino acid residues;
Z²³, Z²⁴, Z^{23′}, and Z^{24′} are each an amino acid residue selected from histidine (H), tyrosine (Y), phenylalanine (F), and tryptophan (W);
X²¹ and X^{21′} are each a tripeptide region consisting of one to six amino acid residues, each of which is independently selected from: histidine (H), lysine (K), arginine (R), alanine (A), cysteine (C), and methionine (M), and one amino acid residue is selected from leucine (L), isoleucine (I), methionine (M), and valine (V);
X²² and X^{22′} are each independently selected from glycine (G), alanine (A), valine (V), leucine (L), isoleucine (I), alloisoleucine, methionine (M), serine (S), and threonine (T);
X²³ and X^{23′} are each an amino acid residue selected from asparagine (N), glutamine (Q), serine (S), and threonine (T);
R²¹ is selected at each occurrence from hydrogen, —C(O)C₁₋₄ alkyl, and L-R²³;
R²² is selected at each occurrence from hydrogen and B—R²³, wherein at least one occurrence of R²¹ or R²² is selected from —C(O)C₁₋₄ alkyl and B—R²³;
   wherein B is absent or an organic linker, and R²³ is a label or a water-soluble oligomer;
wherein the amino acid residues of X²¹, X²², X²³, Z²¹, Z²², Z²³ and Z²⁴ are L-amino acid residues, and the amino acid residues of X^{21′}, X^{22′}, X^{23′}, Z^{21′}, Z^{22′}, Z^{23′} and Z^{24′} are D-amino acid residues.

In some embodiments of the oligopeptide or peptidomimetic of Formula (IIIa) or (IIIb), J²⁰ is a linker region consisting of two to five amino acid residues. In some embodiments, J²⁰ is a linker region consisting of two to four amino acid residues. In some embodiments, J²⁰ is a linker region consisting of two to three amino acid residues. In some embodiments, J²⁰ is a linker region consisting of two amino acid residues. In some embodiment, J²⁰ comprises at least one unnatural amino acid residue.

In some embodiments of the oligopeptide or peptidomimetic of Formula (IIIa) or (IIIb),

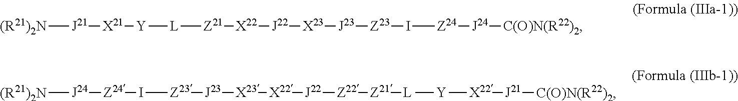

or pharmaceutically acceptable salt thereof, wherein
J²² and J²³ are each an optional linker region consisting of one to three amino acid residues, wherein J²² and J²³ comprise the same number of amino acid residues;
Z²¹, Z^{21′}, Z²² and Z^{22′} are each independently selected from: glycine (G), alanine (A), and serine (S), wherein the amino acid residues of Z²¹ and Z²² are L-amino acid residues, and the amino acid residues of Z^{21′} and Z^{22′} are D-amino acid residues.

In some embodiments of the oligopeptide or peptidomimetic of Formula (IIIa), (IIIb), (IIIa-1), or (IIIb-1), Z²¹, Z^{21′}, Z²², and Z^{22′} are each independently selected from glycine (G) and alanine (A).

In some embodiments of the oligopeptide or peptidomimetic of Formula (IIIa), (IIIb), (IIIa-1), or (IIIb-1), Z²¹ and Z^{21′} are each independently selected from glycine (G), alanine (A), and serine (S). In some embodiments, Z²¹ and Z^{21′} are each independently selected from glycine (G) and alanine (A). In some embodiments, Z²¹ and Z^{21′} are each independently selected from glycine (G) and serine (S). In some embodiments, Z²¹ and Z^{21′} are each independently selected from alanine (A) and serine (S). In some embodiments, Z²¹ and Z^{21′} are each glycine (G). In some embodiments, Z²¹ and Z^{21′} are each alanine (A). In some embodiments, Z²¹ and Z^{21′} are each serine (S).

In some embodiments of the oligopeptide or peptidomimetic of Formula (IIIa), (IIIb), (IIIa-1), or (IIIb-1), Z²² and Z^{22′} are each independently selected from glycine (G), alanine (A), and serine (S). In some embodiments, Z²² and Z^{22′} are each independently selected from glycine (G) and alanine (A). In some embodiments, Z²² and Z^{22′} are each independently selected from glycine (G) and serine (S). In some embodiments Z²² and Z^{22′} are each independently selected from alanine (A) and serine (S). In some embodiments, Z²² and Z^{22′} are each glycine (G). In some embodiments, Z²² and Z^{22′} are each alanine (A). In some embodiments Z²² and Z^{22′} are each serine (S).

In some embodiments of the oligopeptide or peptidomimetic of Formula (IIIa), (IIIb), (IIIa-1), or (IIIb-1), at least one of Z²¹, Z^{21′}, Z²², and Z^{22′} is an unnatural amino acid residue. In some embodiments, at least one of Z²¹, Z^{21′}, Z²², and Z^{22′} is an unnatural amino acid residue comprising a peptide bond isostere.

In some embodiments of the oligopeptide or peptidomimetic of Formula (IIIa), (IIIb), (IIIa-1), or (IIIb-1), Z²³, Z^{23′}, Z²⁴, and Z^{24′} are each independently an amino acid residue selected from histidine (H), tyrosine (Y), and phenylalanine (F). In some embodiments, Z²³, Z^{23′}, Z²⁴, and Z^{24′} are each independently an amino acid residue selected from tyrosine (Y), phenylalanine (F), and tryptophan (W). In some embodiments, Z²³, Z^{23′}, Z²⁴, and Z^{24′} are each independently an amino acid residue selected from tyrosine (Y) and phenylalanine (F). In some embodiments, Z²³, Z^{23′}, Z²⁴, and Z^{24′} are each tyrosine (Y). In some embodiments, Z²³, Z^{23′}, Z²⁴, and Z^{24′} are each phenylalanine (F).

In some embodiments of the oligopeptide or peptidomimetic of Formula (IIIa), (IIIb), (IIIa-1), or (IIIb-1), Z²³ and Z^{23′} are each an amino acid residue selected from histidine (H), tyrosine (Y), phenylalanine (F), and tryptophan (W). In some embodiments, Z²³ and Z^{23′} are each an amino acid residue selected from histidine (H), tyrosine (Y), and phenylalanine (F). In some embodiments, Z²³ and Z^{23′} are each an amino acid residue selected from tyrosine (Y), phenylalanine (F), and tryptophan (W). In some embodiments, Z²³ and Z^{23′} are each an amino acid residue selected from tyrosine (Y) and phenylalanine (F). In some embodiments, Z³ and Z^{3′} are each tyrosine (Y). In some embodiments, Z²³ and Z^{23′} are each phenylalanine (F).

In some embodiments of the oligopeptide or peptidomimetic of Formula (IIIa), (IIIb), (IIIa-1), or (IIIb-1), Z²⁴ and Z^{24′} are each an amino acid residue selected from histidine (H), tyrosine (Y), phenylalanine (F), and tryptophan (W). In some embodiments, Z²⁴ and Z^{24′} are each an amino acid residue selected from histidine (H), tyrosine (Y), and phenylalanine (F). In some embodiments, Z²⁴ and Z^{24′} are each an amino acid residue selected from tyrosine (Y), phenylalanine (F), and tryptophan (W). In some embodiments, Z²⁴ and Z^{24′} are each an amino acid residue selected from tyrosine (Y) and phenylalanine (F). In some embodiments, Z²⁴ and Z^{24′} are each tyrosine (Y). In some embodiments, Z²⁴ and Z^{24′} are each phenylalanine (F).

In some embodiments of the oligopeptide or peptidomimetic of Formula (IIIa), (IIIb), (IIIa-1), or (IIIb-1), at least one of Z²³, Z^{23′}, Z²⁴, and Z^{24′} is an unnatural amino acid residue. In some embodiments, at least one of Z²³, Z^{23′}, Z²⁴, and Z^{24′} is an unnatural amino acid residue comprising a peptide bond isostere.

In some embodiments of the oligopeptide or peptidomimetic of Formula (IIIa), (IIIb), (IIIa-1), or (IIIb-1), X²¹ and X^{21′} are each a tripeptide region consisting of three amino acid residues, wherein at least one amino acid residue is selected from histidine (H), lysine (K), arginine (R), and cysteine (C). In some embodiments, X²¹ and X^{21′} are each a tripeptide region consisting of three amino acid residues, wherein at least one amino acid residue is selected from histidine (H), lysine (K), and arginine (R). In some embodiments, X²¹ and X^{21′} are each a tripeptide region consisting of three amino acid residues, wherein at least one amino acid residue is selected from histidine (H) and lysine (K). In some embodiments, X²¹ and X^{21′} are each a tripeptide region consisting of three amino acid residues, wherein at least one amino acid residue is selected from histidine (H) and arginine (R). In some embodiments, X²¹ and X^{21′} are each a tripeptide region consisting of three amino acid residues, wherein at least one amino acid residue is selected from histidine (H).

In some embodiments, X²¹ and X^{21′} are each a tripeptide region consisting of three amino acid residues, wherein at least two amino acid residue is selected from histidine (H), lysine (K), and arginine (R). In some embodiments, X²¹ and X^{21′} are each a tripeptide region consisting of three amino acid residues, wherein at least two amino acid residue is selected from histidine (H) and lysine (K). In some embodiments, X²¹ and X^{21′} are each a tripeptide region consisting of three amino acid residues, wherein at least two amino acid residue is selected from histidine (H) and arginine (R). In some embodiments, X²¹ and X^{21′} are each a tripeptide region consisting of three amino acid residues, wherein at least two amino acid residue is selected from histidine (H).

In some embodiments of the oligopeptide or peptidomimetic of Formula (IIIa), (IIIb), (IIIa-1), or (IIIb-1), at least two consecutive amino acid residues of X²¹ and X^{21′} are selected from histidine (H), lysine (K), and arginine (R). In some embodiments, X²¹ and X^{21′} are each a tripeptide region consisting of three amino acid residues, wherein at least two consecutive amino acid residue is selected from histidine (H) and lysine (K).

In some embodiments, X²¹ and X^{21′} are each a tripeptide region consisting of three amino acid residues, wherein at least two consecutive amino acid residue is selected from histidine (H) and arginine (R). In some embodiments, X²¹ and X^{21′} are each a tripeptide region consisting of three amino acid residues, wherein at least two consecutive amino acid residue is selected from histidine (H).

In some embodiments of the oligopeptide or peptidomimetic of Formula (IIIa), (IIIb), (IIIa-1), or (IIIb-1), X²¹ and X^{21′} are each a tripeptide region consisting of three amino acid residues, wherein one amino acid residue is selected from leucine (L), isoleucine (I), and valine (V). In some embodiments, X²¹ and X^{21′} are each a tripeptide region consisting of three amino acid residues, wherein one amino acid residue is selected from isoleucine (I) and valine (V). In some embodiments, X²¹ and X^{21′} are each a tripeptide region consisting of three amino acid residues, wherein one amino acid residue is selected from leucine (L) and isoleucine (I). In some embodiments, X²¹ and X^{21′} are each a tripeptide region consisting of three amino acid residues, wherein one amino acid residue is isoleucine (I).

In some embodiments of the oligopeptide or peptidomimetic of Formula (IIIa), (IIIb), (IIIa-1), or (IIIb-1), X²¹ and X^{21′} consist of the following amino acid sequence: —H—H—I—.

In some embodiments of the oligopeptide or peptidomimetic of Formula (IIIa), (IIIb), (IIIa-1), or (IIIb-1), X²² and X^{22′} are each an amino acid residue selected from glycine (G), alanine (A), valine (V), leucine (L), isoleucine (I), and methionine (M). In some embodiments, X²² and X^{22′} are each an amino acid residue selected from alanine (A), valine (V), leucine (L), isoleucine (I), and methionine (M). In some embodiments, X²² and X^{22′} are each an amino acid residue selected from valine (V), leucine (L), isoleucine (I), and methionine (M). In some embodiments, X²² and X^{22′} are each an amino acid residue selected from alanine (A), valine (V), leucine (L), and isoleucine (I). In some embodiments, X²² and X^{22′} are each an amino acid residue selected from valine (V), leucine (L), and isoleucine (I). In some embodiments, X²² and X^{22′} are each an amino acid residue selected from valine (V) and isoleucine (I). In some embodiments, X²² and X^{22′} are each an amino acid residue selected from valine (V) and leucine (L). In some embodiments X²² and X^{22′} are each valine (V).

In some embodiments of the oligopeptide or peptidomimetic of Formula (IIIa), (IIIb), (IIIa-1), or (IIIb-1), X²³ and X^{23′} are each an amino acid residue selected from asparagine (N), glutamine (Q), serine (S), and threonine (T). In some embodiments, X²³ and X^{23′} are each an amino acid residue selected from asparagine (N), serine (S), and threonine (T). In some embodiments, X²³ and X^{23′} are each an amino acid residue selected from asparagine (N), glutamine (Q), and threonine (T). In some embodiments, X²³ and X^{23′} are each an amino acid residue selected from asparagine (N), glutamine (Q), and serine (S). In some embodiments, X²³ and X^{23′} are each an amino acid residue selected from asparagine (N) and serine (S). In some embodiments, X²³ and X^{23′} are each an amino acid residue selected from asparagine (N) and threonine (T). In some embodiments, X²³ and X^{23′} are each an amino acid residue selected from asparagine (N) and glutamine (Q). In some embodiments, X²³ and X^{23′} are each asparagine (N).

In some embodiments of the oligopeptide or peptidomimetic of Formula (IIIa), (IIIb), (IIIa-1), or (IIIb-1), J²¹, J²², J²³, and J²⁴ are each optional linker regions consisting of one to five amino acid residues. In some embodiments, J²¹, J²², J²³, and J²⁴ are each optional linker regions consisting of one to four amino acid residues. In some embodiments, J²¹, J²², J²³, and J²⁴ are each optional linker regions consisting of one to three amino acid residues. In some embodiments J²¹, J²², J²³, and J²⁴ are each optional linker regions consisting of one to two amino acid residues. In some embodiments, J²¹, J²², J²³, and J²⁴ are each optional linker regions consisting of one amino acid residue.

In some embodiments of the oligopeptide or peptidomimetic of Formula (IIIa), (IIIb), (IIIa-1), or (IIIb-1), J²¹, J²², J²³, and J²⁴ are each absent. In some embodiments, J²¹, J²², and J²³ are each absent. In some embodiments, J²², J²³, and J²⁴ are each absent.

In some embodiments, J²¹, J²², and J²⁴ are each absent. In some embodiments, J²¹, J²³, and J²⁴ are each absent. In some embodiments, J²¹ and J²² are each absent. In some embodiments, J²¹ and J²³ are each absent. J¹¹ and J¹⁴ are each absent. In some embodiments, J²² and J²³ are each absent. In some embodiments, J²² and J²⁴ are each absent. In some embodiments, J²³ and J²⁴ are each absent. In some embodiments, J²¹ is absent. In some embodiments, J²² is absent. In some embodiments, J²³ is absent. In some embodiments, J²⁴ is absent.

In some embodiments of the oligopeptide or peptidomimetic of Formula (IIIa), (IIIb), (IIIa-1), or (IIIb-1), at least one of J²¹, J²², J²³, and J²⁴ is a peptide linker region consisting of one to six amino acid residues. In some embodiments, at least one of J²¹, J²², J²³, and J²⁴ is a peptide linker region consisting of one to five amino acid residues.

In some embodiments, at least one of J²¹, J²², J²³, and J²⁴ is a peptide linker region consisting of one to four amino acid residues. In some embodiments, at least one of J²¹, J²², J²³, and J²⁴ is a peptide linker region consisting of one to three amino acid residues.

In some embodiments, at least one of J²¹, J²², J²³, and J²⁴ is a peptide linker region consisting of one to two amino acid residues.

In some embodiments of the oligopeptide or peptidomimetic of Formula (IIIa), (IIIb), (IIIa-1), or (IIIb-1), J²² and J²³ comprise the same number of amino acid residues.

In some embodiments, J²² and J²³ comprise a different number of amino acid residues. In some embodiment, J²² and J²³ each consist of one to three amino acid residues. In some embodiment, J²² and J²³ each consist of one to two amino acid residues. In some embodiment, J²² and J²³ each consist of two to three amino acid residues. In some embodiment, J²² and J²³ each consist of one amino acid residue. In some embodiment, J²² and J²³ each consist of two amino acid residues. In some embodiment, J²² and J²³ each consist of three amino acid residues.

In some embodiments of the oligopeptide or peptidomimetic of Formula (IIIa), (IIIb), (IIIa-1), or (IIIb-1), at least one of J²¹, J²², J²³, and J²⁴ a peptide linker region comprising at least one unnatural amino acid residue. In some embodiments, at least one of J²¹, J²², J²³, and J²⁴ is a peptide linker region comprising at least two unnatural amino acid residue. In some embodiments, at least three of J²¹, J²², J²³, and J²⁴ is a peptide linker region comprising at least two unnatural amino acid residue.

In some embodiments of the oligopeptide or peptidomimetic of Formula (IIIa), (IIIb), (IIIa-1), or (IIIb-1), R²¹ is selected at each occurrence from hydrogen and B—R²³. In some embodiments, at least one R²¹ is hydrogen. In some embodiments, each occurrence of R²¹ is hydrogen.

In some embodiments of the oligopeptide or peptidomimetic of Formula (IIIa), (IIIb), (IIIa-1), or (IIIb-1), R²² is selected at each occurrence from hydrogen and B—R²³.

In some embodiments, at least one R²² is hydrogen. In some embodiments, each occurrence of R²² is hydrogen. In some embodiments, each occurrence of R²¹ and R²² is hydrogen. In some embodiments, at least one occurrence of R²¹ and R²² is B—R²³.

In some embodiments, B is absent or an organic linker, and R²³ is a label or a water-soluble oligomer. In some embodiments, B is absent. In some embodiments, B is an organic linker. In some embodiments, B is an organic linker comprising polyethylene glycol (PEG). In some embodiments, R²³ is a label. In some embodiments, R²³ is a fluorescent label. In some embodiments, R²³ is a label. In some embodiments, R²³ is a water-soluble oligomer. In some embodiments, R²³ is a water-soluble oligomer comprising PEG.

In some embodiments of the oligopeptide or peptidomimetic of Formula (IIIa), (IIIb), (IIIa-1), or (IIIb-1), the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

In some embodiments of the oligopeptide or peptidomimetic of Formula (IIIa), (IIIb), (IIIa-1), or (IIIb-1), at least one amino acid residue of Z¹¹, Z^{11′}, Z¹², Z^{12′}, Z¹³, Z^{13′}, Z¹⁴, and Z^{14′} is an unnatural amino acid residue. In some embodiments, at least one amino acid residue of Z¹³, Z^{13′}, Z¹⁴, and Z^{14′} is an unnatural amino acid residue having a side chain comprising a substituted phenyl ring.

In some embodiments, the oligopeptide or peptidomimetic is represented by Formula (IIIa) or a pharmaceutically acceptable salt thereof. In some embodiments, the oligopeptide or peptidomimetic is represented by Formula (IIIb) or a pharmaceutically acceptable salt thereof. In some embodiments, the oligopeptide or peptidomimetic is represented by Formula (IIIa-1) or a pharmaceutically acceptable salt thereof. In some embodiments, the oligopeptide or peptidomimetic is represented by Formula (IIIb-1) or a pharmaceutically acceptable salt thereof.

In some embodiments of the oligopeptide or peptidomimetic of Formula (Ia), (Ib), (Ia-1), (Ib-1), (Ia-2), (Ib-2), (Ia-3), (Ib-3), (Ia-4), (Ib-4), (IIa), (IIb), (IIa-1), (IIb-1), (IIa-2), (IIb-2), (IIa-3), (IIb-3), (IIa-4), (IIb-4), (IIIa), (IIIb), (IIIa-1), or (IIIb-1), or a pharmaceutically acceptable salt thereof, the pharmaceutically acceptable salt comprises an acetate salt.

An oligopeptide or peptidomimetic consisting of between ten and twenty amino acid residues, the oligopeptide comprising:
(i) a first region consisting of the amino acid sequence Y-L-G-A;
(ii) a second region consisting of the amino acid sequence Y-I-Y; and
(iii) a linker region comprising a plurality of amino acid residues connecting (i) to (ii);
wherein the oligopeptide or peptidomimetic comprises a C-terminal amide;
provided that the oligopeptide is not NH₂—HHIYLGAVNYIY-C(O)NH₂.

In some embodiments, at least one amino acid residue of the oligopeptide or peptidomimetic is an L-amino acid residue. In some embodiments, at least five amino acid residues of the oligopeptide or peptidomimetic are L-amino acid residues. In some embodiments, at least ten amino acid residues of the oligopeptide or peptidomimetic are L-amino acid residues. In some embodiments, all amino acid residues of the oligopeptide or peptidomimetic are L-amino acid residues.

In some embodiments, the linker region comprises from 1 to 6 amino acid residues. In some embodiments, the linker region comprises from 1 to 5 amino acid residues. In some embodiments, the linker region comprises from 1 to 4 amino acid residues. In some embodiments, the linker region comprises from 1 to 3 amino acid residues. In some embodiments, the linker region comprises from 1 to 2 amino acid residues.

In some embodiments, the linker region comprises from 2 to 6 amino acid residues. In some embodiments, the linker region comprises from 2 to 5 amino acid residues. In some embodiments, the linker region comprises from 2 to 4 amino acid residues. In some embodiments, the linker region comprises from 2 to 3 amino acid residues. In some embodiments, the linker region comprises 2 amino acid residues.

In some embodiments, the linker region consists of from 1 to 6 amino acid residues. In some embodiments, the linker region consists of from 1 to 5 amino acid residues. In some embodiments, the linker region consists of from 1 to 4 amino acid residues. In some embodiments, the linker region consists of from 1 to 3 amino acid residues. In some embodiments, the linker region consists of from 1 to 2 amino acid residues.

In some embodiments, the linker region consists of from 2 to 6 amino acid residues. In some embodiments, the linker region consists of from 2 to 5 amino acid residues. In some embodiments, the linker region consists of from 2 to 4 amino acid residues. In some embodiments, the linker region consists of from 2 to 3 amino acid residues. In some embodiments, the linker region consists of 2 amino acid residues. In some embodiments, the linker region comprises at least one of unnatural amino acid residue.

In some embodiments, the oligopeptide or peptidomimetic comprises between 10 and 19 amino acid residues. In some embodiments, the oligopeptide or peptidomimetic comprises between 10 and 18 amino acid residues. In some embodiments, the oligopeptide or peptidomimetic comprises between 10 and 17 amino acid residues. In some embodiments, the oligopeptide or peptidomimetic comprises between 10 and 16 amino acid residues. In some embodiments, the oligopeptide or peptidomimetic comprises between 10 and 15 amino acid residues. In some embodiments, the oligopeptide or peptidomimetic comprises between 10 and 14 amino acid residues. In some embodiments, the oligopeptide or peptidomimetic comprises between 10 and 13 amino acid residues. In some embodiments, the oligopeptide or peptidomimetic comprises between 10 and 12 amino acid residues. In some embodiments, the oligopeptide or peptidomimetic comprises between 10 and 11 amino acid residues.

In some embodiments, the oligopeptide or peptidomimetic comprises 10 amino acid residues. In some embodiments, the oligopeptide or peptidomimetic comprises 11 amino acid residues. In some embodiments, the oligopeptide or peptidomimetic comprises 12 amino acid residues. In some embodiments, the oligopeptide or peptidomimetic comprises 13 amino acid residues. In some embodiments, the oligopeptide or peptidomimetic comprises 14 amino acid residues. In some embodiments, the oligopeptide or peptidomimetic comprises 15 amino acid residues. In some embodiments, the oligopeptide or peptidomimetic comprises 16 amino acid residues. In some embodiments, the oligopeptide or peptidomimetic comprises 17 amino acid residues. In some embodiments, the oligopeptide or peptidomimetic comprises 18 amino acid residues. In some embodiments, the oligopeptide or peptidomimetic comprises 19 amino acid residues. In some embodiments, the oligopeptide or peptidomimetic comprises 20 amino acid residues.

In some embodiments, the oligopeptide or peptidomimetic consists of 10 amino acid residues. In some embodiments, the oligopeptide or peptidomimetic consists of 11 amino acid residues. In some embodiments, the oligopeptide or peptidomimetic consists of 12 amino acid residues. In some embodiments, the oligopeptide or peptidomimetic consists of 13 amino acid residues. In some embodiments, the oligopeptide or peptidomimetic consists of 14 amino acid residues. In some embodiments, the oligopeptide or peptidomimetic consists of 15 amino acid residues. In some embodiments, the oligopeptide or peptidomimetic consists of 16 amino acid residues. In some embodiments, the oligopeptide or peptidomimetic consists of 17 amino acid residues. In some embodiments, the oligopeptide or peptidomimetic consists of 18 amino acid residues. In some embodiments, the oligopeptide or peptidomimetic consists of 19 amino acid residues. In some embodiments, the oligopeptide or peptidomimetic consists of 20 amino acid residues.

In some embodiments, at least one of the amino acid residues of the first region is replaced with a conservative amino acid residue substitution. In some embodiments, at least one of the amino acid residues of the second region is replaced with a conservative amino acid residue substitution.

In some embodiments, the oligopeptide or peptidomimetic comprises at least one unnatural amino acid residue. In some embodiments, at least one of the amino acid residues of the first region is replaced with an unnatural amino acid residue substitution.

In some embodiments, at least one of the amino acid residues of the second region is replaced with an unnatural amino acid residue substitution.

In some embodiments, the oligopeptide or peptidomimetic comprises at least one peptidomimetic amino acid residue. In some embodiments, the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

In some embodiments, the oligopeptide or peptidomimetic is an oligopeptide or peptidomimetic of Table 1, below:

TABLE 1

| Cmpd. | | Amino acid |
|---|---|---|
| No. | Structure | chirality |
| 1 | NH₂-AHIYLGAVNYIY-C(O)NH₂ | L |
| 2 | NH₂-HAIYLGAVNYIY-C(O)NH₂ | L |
| 3 | NH₂-HHIYLAAVNYIY-C(O)NH₂ | L |
| 4 | NH₂-HHIYLGAANYIY-C(O)NH₂ | L |
| 5 | NH₂-[D]-(Ac)-YIYNVAGLYIHH-C(O)NH₂ | D |
| 6 | NH₂-HHIAAGAVNYIY-C(O)NH₂ | L |
| 7 | NH₂-HHIYLGGGGGNYIY-C(O)NH₂ | L |
| 8 | NH₂-HHIYLGSSSSNYIY-C(O)NH₂ | L |
| 9 | NH₂-KKIYLGAVNYIY-C(O)NH₂ | L |
| 10 | NH₂-KHIYLGAVNYIY-C(O)NH₂ | L |
| 11 | NH₂-HKIYLGAVNYIY-C(O)NH₂ | L |
| 12 | NH₂-HHIAAGAVNYIY-C(O)NH₂ | L |
| 13 | NH₂-HHIYLGAVNYIY-C(O)N(H)-PEG | L |
| 14 | NH₂-HHIYLGAVNFIF-C(O)NH₂ | L |
| 15 | NH₂-HCIYLGATNYIYC-C(O)NH₂ | L |
| 16 | FITC-Ahx-N(H)-HHIYLGAVNYIY-C(O)NH₂ | L |
| 17 | NH₂-HHIYLGAVNYIY-C(O)N(H)-Ahx-FITC | L |

### Pharmaceutical Compositions

Certain embodiments relate to compositions that include the described Fas inhibitor(s), a derivative, fragment, a pharmaceutically acceptable salt thereof, or a gene therapy encoding the described Fas inhibitor in an amount effective to inhibit Fas signaling.

The composition may be a “pharmaceutical composition,” a “pharmaceutical preparation,” or a “pharmaceutical formulation.”

As used herein, the term “pharmaceutical composition” refers to the combination of one or more pharmaceutical agents (e.g., Fas inhibitor) with one or more carriers, inert or active, making the composition especially suitable for diagnostic or therapeutic use in vitro, in vivo or ex vivo. A pharmaceutical composition comprises the physical entity that is administered to a subject, and may take the form of a solid, semi-solid or liquid dosage form, such as tablet, capsule, orally-disintegrating tablet, pill, powder, suppository, solution, elixir, syrup, suspension, cream, lozenge, paste, spray, etc. A pharmaceutical composition may comprise a single pharmaceutical formulation (e.g., extended release, immediate release, delayed release, nanoparticulate, etc.) or multiple formulations (e.g., immediate release and delayed release, nanoparticulate and non-nanoparticulate, etc.).

As used herein, the terms “pharmaceutical preparation” or “pharmaceutical formulation” refer to at least one, but may be two, three or more, pharmaceutical agent(s) (e.g., Fas inhibitor, e.g., Met, Met-12 or Compound 1) in combination with one or more additional components that assist in rendering the pharmaceutical agent(s) suitable for achieving the desired effect upon administration to a subject. The pharmaceutical formulation may include one or more additives, for example pharmaceutically acceptable excipients, carriers, penetration enhancers, coatings, stabilizers, buffers, acids, bases, or other materials physically associated with the pharmaceutical agent to enhance the administration, release (e.g., timing of release), deliverability, bioavailability, effectiveness, etc. of the dosage form. The formulation may be, for example, a liquid, a suspension, a solid, a nanoparticle, emulsion, micelle, ointment, gel, emulsion, coating, etc. A pharmaceutical formulation may contain a single pharmaceutical agent (e.g., Met, Met-12 or Compound 1) or multiple pharmaceutical agents. A pharmaceutical composition may contain a single pharmaceutical formulation or multiple pharmaceutical formulations. In some embodiments, a pharmaceutical agent (e.g., Met, Met-12 or Compound 1) is formulated for a particular mode of administration (e.g., ocular administration (e.g., intravitreal administration, etc.), etc.). A pharmaceutical formulation is sterile, non-pyrogenic and non-toxic to the subject. The terms “pharmaceutical composition” and “pharmaceutical formulation” may be used interchangeably.

Certain embodiments, relate to compositions that include the described Fas inhibitor, a derivative, or a pharmaceutically acceptable salt thereof; and a pharmaceutically acceptable additive. The additive may be selected from carriers, excipients, disintegrators or disintegrating aids, binders, lubricants, coating agents, pigments, diluents, bases, dissolving agents or solubilizers, isotonic agents, pH regulators, stabilizers, propellants, adhesives, and other additives known in the art.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used.

PEG may be used (even apart from its use in derivatizing the protein or analog). Dextrans, such as cyclodextran, may be used. Bile salts and other related enhancers may be used.

Cellulose and cellulose derivatives may be used. Amino acids may be used, such as use in a buffer formulation.

For further examples of carriers, stabilizers and adjuvants see, e.g., Martin, Remington's Pharmaceutical Sciences, 15th Ed., Mack Publ. Co., Easton, Pa. [1975]; herein incorporated by reference in its entirety.

Also, the use of liposomes, microcapsules or microspheres, inclusion complexes, or other types of carriers known in the art, is contemplated.

In certain embodiments, the composition may include at least one non-ionic surfactant. Examples of non-ionic surfactants include Polysorbate 80, Polysorbate 20, Poloxamer 407, and Tyloxapol.

The composition may be in any form suitable for administration to a subject, e.g., solution, pill, ointment, suspension, eye drops, gel, cream, foam, spray, liniment, and powder. As used herein, the term “administration” refers to the act of giving a drug, prodrug, or other agent, or therapeutic treatment (e.g., Fas inhibitor and/or compositions thereof described herein) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, intravitreally, periocularly, etc.) and the like. Implantable sustained release forms/formulations are also contemplated.

The compositions and methods described herein are particularly applicable for human subjects at risk for or suffering from inflammation-mediated and/or complement-mediated disease or condition, such as retinal disease (e.g., glaucoma, retinal detachment, AMD (dry and wet), diabetic retinopathy, Uveitis, retinal vein occlusion, retinitis pigmentosa or NAION), immunological disease, cancer, amyloid disease (e.g., Alzheimer's disease, type-2 diabetes, Huntington's disease, ALS, or Parkinson's disease), autoimmune disease (e.g., allergy, lupus, or rheumatoid arthritis), an injury caused by ischemia or reperfusion (e.g., stroke), neurodegeneration, and diseases of the central nervous system. The etiology of the disease or condition, itself, may or may not be Fas-mediated, but Fas-mediated signaling through one or more signaling pathways accelerates or amplifies disease symptoms and/or severity.

The compositions for topical use could be in any form deemed suitable by the person skilled in the art to be applied directly on the ocular surface, like e.g., solution, ointment, suspension, eye drops, gel, cream, foam, spray, liniment, powder.

The Fas inhibitor or a composition thereof may be administered daily (once, twice, 3 times, 4 times/day, etc.), every other day, every 3 days, weekly, biweekly, monthly, bimonthly, or tri-monthly, etc.

The described Fas inhibitors or compositions thereof may be administered in an amount effective to inhibit Fas and/or Fas signaling. The term “an amount effective” means an amount of a drug or agent (e.g., Compound 1) or its' formulation effective to facilitate a desired therapeutic effect (e.g., inhibition of Fas signaling) in a particular class of subjects (e.g., infant, child, adolescent, adult). U.S. Food and Drug Administration (FDA) recommended dosages are indicative of a therapeutic dose. For example, in the context of this application, the desired therapeutic effect may be preventing or treating inflammation-mediated and/or complement-mediated disease or condition or limiting the severity of inflammation-mediated and/or complement-mediated disease or condition.

For example, an effective amount may be a daily dose of Fas inhibitor in a range, e.g., from about 1 ng to about 1 mg.

In one embodiment, the composition is in the form of eye drops and the described Fas inhibitor is in a concentration between 0.000001% w/v and 2% w/v.

In certain embodiments, compositions comprise one or more additives, such as carriers, diluents and/or excipients suitable for preparing, e.g., ophthalmic compositions. Suitable for preparing ophthalmic compositions are all carriers, diluents or excipients tolerated by the eye. Examples of excipients that may be used in said compositions are Polysorbate 80, polyethylene glycol (e.g., PEG200, PEG400) dextran and the like.

The compositions may comprise carriers for improving the Fas inhibitor's bioavailability by increasing corneal permeability, like e.g. dimethyl sulfoxide, membrane phospholipids and surfactants.

In certain embodiment, such compositions may also comprise carriers apt to increase bioavailability, stability and tolerability of the active principle. For instance, viscosity-increasing agents such as hyaluronic acid, methylcellulose, polyvinyl alcohol, polyvinyl pyrrolidone, etc. may be used.

To prevent contaminations, the described compositions could comprise one or more preservatives having antimicrobial activity, like e.g. benzalchonium chloride (shortened in BAK).

### Uses and Methods

In certain embodiments, the described Fas inhibitors may be used for preventing, treating or ameliorating an inflammation-mediated and/or complement-mediated disease or condition in a subject.

Examples of diseases or conditions that may be treated with the described Fas inhibitors include, e.g., retinal disease (e.g., glaucoma, retinal detachment, AMD (dry and wet), diabetic retinopathy, Uveitis, retinal vein occlusion, inherited retinal degeneration diseases including retinitis pigmentosa, or NAION), immunological disease, cancer, amyloid disease (e.g., Alzheimer's disease, type-2 diabetes, Huntington's disease, ALS, or Parkinson's disease), traumatic injury (e.g. traumatic brain injury), autoimmune disease (e.g., allergy, lupus, or rheumatoid arthritis), an injury caused by ischemia or reperfusion (e.g., stroke), neurodegeneration, and diseases of the central nervous system (e.g., neuropathies and demyelinating diseases such as multiple sclerosis and inflammatory demyelinating diseases).

Surprisingly, without being bound by the mechanism of action, it was discovered that the inhibition of Fas/Fas signaling results in at least one of the following: reduction of expression or concentration of at least one Fas-mediated inflammation-related gene or protein; reduction of expression or concentration of at least one Fas-mediated complement-related gene or protein, including complement component 3 (C3) and complement component 1q (C1q); reduction of gene or protein expression or concentration of Caspase 8; reduction of gene or protein expression or concentration of one or more components of the inflammasome, including NLRP3 and NLRP2; reduction of gene or protein expression or concentration of one or more C—X—C motif chemokines, including CXCL2 (MIP-2a) and CXCL10 (IP-10); reduction of gene or protein expression or concentration of one or more C-X3-C motif chemokines, including CX3CL1 (fractalkine); reduction of gene or protein expression or concentration of one or more C—C motif chemokines, including CCL2 (MCP-1), CCL3 (MIP-1α), and CCL4 (MIP-1); reduction of gene or protein expression or concentration of toll-like receptor 4 (TLR4); reduction of gene or protein expression or concentration of one or more interleukin cytokines, including IL-1β, IL-18, and IL-6; reduction of gene or protein expression or concentration of one or more TNF superfamily cytokines, including TNFα; reduction of Fas-mediated Müller cell activation as indicated by reduced GFAP gene or protein expression or concentration; or increase of expression or concentration or prevent the reduction of expression or concentration of at least one pro-survival gene or protein (e.g., cFLIP). The term “Fas-mediated” means involving or depending on the Fas receptor and/or its activation.

As such, certain embodiments relate to a method for preventing, treating, or ameliorating inflammation-mediated and/or complement-mediated disease or condition in a subject including administering to the subject the described Fas inhibitor or a derivative thereof, or a fragment thereof, or a gene therapy encoding the Fas inhibitor in an amount effective to inhibit Fas and/or Fas signaling, and thereby ameliorate or prevent the disease or condition in the subject, wherein the inhibition of Fas and/or Fas signaling results in at least one (or at least two, or at least three, etc., or all) of the following: reduction of expression or concentration of at least one Fas-mediated inflammation-related gene or protein (e.g. α, IL-1β, IP-10, IL-18, MIP1α, IL-6, GFAP, MIP2, MCP-1, or MIP-1β); reduction of expression or concentration of at least one Fas-mediated complement-related gene or protein (e.g., complement component 3 (C3) and complement component 1q (C1q)); reduction of gene or protein expression or concentration of Caspase 8; reduction of gene or protein expression or concentration of one or more components of the inflammasome (e.g., NLRP3 and NLRP2); reduction of gene or protein expression or concentration of one or more C—X—C motif chemokines (e.g., CXCL2 (MIP-2a) and CXCL10 (IP-10)); reduction of gene or protein expression or concentration of one or more C-X3-C motif chemokines (e.g., CX3CL1 (fractalkine)); reduction of gene or protein expression or concentration of one or more C—C motif chemokines (e.g., CCL2 (MCP-1), CCL3 (MIP-1α), and CCL4 (MIP-1β)); reduction of gene or protein expression or concentration of toll-like receptor 4 (TLR4); reduction of gene or protein expression or concentration of one or more interleukin cytokines (e.g., IL-1β, IL-18, and IL-6); reduction of gene or protein expression or concentration of one or more TNF superfamily cytokines (e.g., TNFα); reduction of Fas-mediated Müller cell activation as indicated by reduced GFAP gene or protein expression or concentration; or increase of expression or concentration or prevent the reduction of expression or concentration of at least one pro-survival gene or protein (e.g., cFLIP). The Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or gene therapy agents encoding the Fas inhibitor. The subject may have or is at risk of having the inflammation-mediated and/or complement-mediated disease or condition

The inflammation-mediated and/or complement-mediated disease or condition may be a retinal disease, immunological disease, cancer, amyloid disease, an injury caused by ischemia or reperfusion, an injury caused by trauma, neurodegeneration, and diseases of the central nervous system. Examples of the amyloid disease include Alzheimer's disease, type-2 diabetes, Huntington's disease, ALS, or Parkinson's disease. An example of the injury by ischemia or reperfusion is stroke. An example of the injury by trauma is traumatic brain injury. Exemplary autoimmune diseases include allergies, lupus, and rheumatoid arthritis. Exemplary retinal diseases include glaucoma, retinal detachment, AMD (dry and wet), diabetic retinopathy, Uveitis, retinal vein occlusion, inherited retinal degeneration including retinitis pigmentosa, and NAION. Examples of diseases of the central nervous system include neuropathy or a demyelinating disease selected from the group consisting of multiple sclerosis and inflammatory demyelinating diseases.

In the described methods, the Fas inhibitor, its derivative, or the pharmaceutically acceptable salt thereof may be administered via an injection.

Another embodiment related to a method for preserving retinal ganglion cells and axon density, or preventing the loss of ganglion cells and axon density in a patient with glaucoma comprising administering to the subject a Fas inhibitor, a derivative thereof, a fragment thereof, a pharmaceutically acceptable salt thereof, or a gene therapy encoding the Fas inhibitor, wherein the preserving or preventing the loss of retinal ganglion cells and axon density, or preventing the loss thereof is due to at least one (or at least two, or all three) of the following: inhibition of microglial/macrophage activation or recruitment; inhibition of at least one of TNF-α, CCL2/MCP-1 or CCL3/MIP-1α gene or protein expression or concentration; or reduction of IL-10 gene or protein expression or protein maturation, wherein the Fas inhibitor is administered to the subject in an amount effective to inhibit Fas signaling. The Fas inhibitor, a derivative thereof, a fragment thereof, a pharmaceutically acceptable salt thereof, or a gene therapy encoding the Fas inhibitor may be administered in a pharmaceutical composition comprising the Fas inhibitor, a derivative thereof, a fragment thereof, a pharmaceutically acceptable salt thereof, or a gene therapy encoding the Fas inhibitor; and a pharmaceutically acceptable additive. The additive may be selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants and adhesives. The composition may be in a form selected from the group consisting of: solution, pill, ointment, suspension, eye drops, gel, cream, foam, spray, liniment, and powder. The administering may be via an injection, wherein the injection is an intravitreal injection, intrathecal, intravenous or periocular injection. The composition may further comprise at least one non-ionic surfactant selected from the group consisting of Polysorbate 80, Polysorbate 20, Poloxamer 407, and Tyloxapol.

The section headings used herein are for organizational purposes only and are not to be construed as limiting the subject matter described.

### EXAMPLES

### Example 1—Synthesis

Synthesis of Compounds 1-17 was performed using standard peptide synthesis protocols, including those disclosed in U.S. Pat. No. 10,829,518, which is incorporated by reference herein in its entirety.

### Example 2—Cell Viability Preservation In Vitro

Compounds (oligopeptides) of the present disclosure were assayed for their ability to effect cell confluence following a 24-hour incubation. Jurkat cells were incubated for 24 hours with varying concentrations of compound (i.e., 200, 66.7, 22.2, 7.4, 2.5, 0.8, 0.3, and 0.1) and with or without recombinant His-tagged FasL along with an anti-His antibody. After the 24-hour incubation, cell confluence was quantified using an S3 Incucyte Imaging System (Sartorius) and a Vi-CELL XR Cell Viability Analyzer (Beckman Coulter) and compared to controls (cells incubated in the absence of compound and cells incubated with a known Fas inhibitor). Analysis of the resulting data demonstrated a preservation of cell confluence for the treated cells compared to the control cells, demonstrating the cyto-protective properties of the oligopeptides of the present disclosure. The lowest concentration that prevented loss of cell confluency is provided in Table 2. Similar cytoprotective activity was observed for other compounds of the present disclosure, including for example, Compounds 1-5 and 12-17.

TABLE 2

| Cmpd. No. | Activity |
|---|---|
| 6 | B |
| 7 | B |
| 8 | B |
| 9 | B |
| 10 | A |
| 11 | B |
| A | A |

A: cell protection at x < 1.0 μM;
B: cell protection at 1.0 < x < 10.0 μM
C: cell protection at x > 10.0 μM

1. An oligopeptide or peptidomimetic of Formula (Ia) or Formula (Ib): 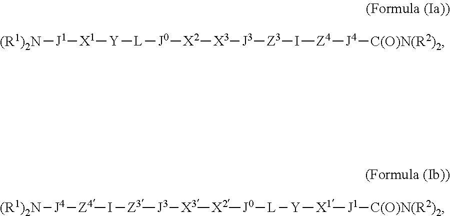
or a pharmaceutically acceptable salt thereof, wherein:
J⁰ is a linker region consisting of two to five amino acid residues;
J¹ and J⁴ are each optional linker regions consisting of one to six amino acid residues;
J³ is an optional linker region consisting of one to three amino acid residues;
Z³, Z⁴, Z^{3′}, and Z^{4′} are each an amino acid residue selected from histidine (H), tyrosine (Y), phenylalanine (F), and tryptophan (W);
X¹ and X^{1′} are each a tripeptide region consisting of three amino acid residues, wherein at least one amino acid residue is selected from histidine (H), lysine (K), arginine (R), alanine (A), cysteine (C), and methionine (M), and one amino acid residue is selected from leucine (L), isoleucine (I), methionine (M), and valine (V);
X² and X^{2′} are each independently selected from (i) and (ii):
(i) when Z³ and Z⁴ are each tyrosine (Y) or when Z^{3′} and Z^{4′} are each tyrosine (Y), X² and X^{2′} are each selected from leucine (L), isoleucine (I), alloisoleucine, methionine (M), serine (S), and threonine (T); or
(ii) when at least one of Z³ and Z⁴ is not tyrosine (Y) or when at least one of Z^{3′} and Z^{4′} is not tyrosine (Y),
X² and X^{2′} are each selected from glycine (G), alanine (A), valine (V), leucine (L), isoleucine (I), alloisoleucine, methionine (M), serine (S), threonine (T);
X³ and X^{3′} are each an amino acid residue selected from asparagine (N), glutamine (Q), serine (S), and threonine (T);
R¹ is selected at each occurrence from hydrogen, —C(O)C₁₋₄ alkyl, and B—R³;
R² is selected at each occurrence from hydrogen and B—R³;
wherein B is absent or an organic linker, and R³ is a label or a water-soluble oligomer;
wherein the amino acid residues of X¹, X², X³, Z³ and Z⁴ are L-amino acid residues, and the amino acid residues of X^{1′}, X^{2′}, X^{3′}, Z^{3′} and Z^{4′} are D-amino acid residues.

2. The oligopeptide or peptidomimetic of claim 1, wherein the oligopeptide or peptidomimetic is represented by Formula (Ia-1) or Formula (Ib-1): 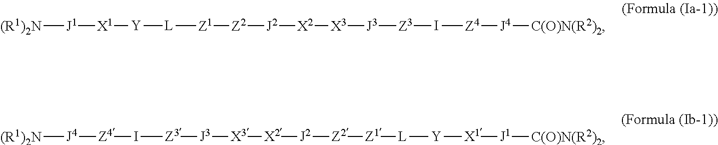
or a pharmaceutically acceptable salt thereof, wherein:
J² and J³ are each an optional linker region consisting of one to three amino acid residues, wherein J² and J³ comprise the same number of amino acid residues; and
Z¹, Z^{1′}, Z², and Z^{2′} are each independently selected from glycine (G), alanine (A), and serine (S), wherein the amino acid residues of Z¹ and Z² are L-amino acid residues, and the amino acid residues of Z^{1′} and Z^{2″} are D-amino acid residues.

3. The oligopeptide or peptidomimetic of claim 2, wherein Z¹, Z^{1′}, Z², and Z^{2′} are each independently selected from glycine (G) and alanine (A).

4. The oligopeptide or peptidomimetic of any one of claim 1 to 3, wherein the oligopeptide or peptidomimetic is represented by Formula (Ia-2) or Formula (Ib-2): 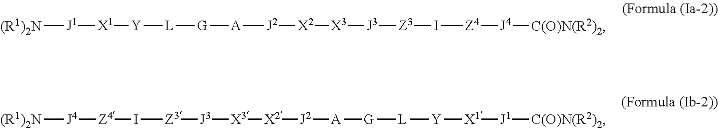
or a pharmaceutically acceptable salt thereof.

5. The oligopeptide or peptidomimetic of any one of claims 1 to 4, Z³, Z⁴, Z^{3′}, and Z^{4′} are each an amino acid residue selected from tyrosine (Y) and phenylalanine (F).

6. The oligopeptide or peptidomimetic of any one of claim 1 to 5, wherein the oligopeptide or peptidomimetic is represented by Formula (Ia-3) or Formula (Ib-3): 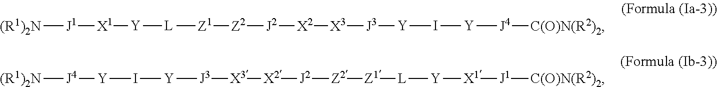
or a pharmaceutically acceptable salt thereof.

7. The oligopeptide or peptidomimetic of any one of claims 1 to 6, wherein the oligopeptide or peptidomimetic is represented by Formula (Ia-4) or Formula (Ib-4): 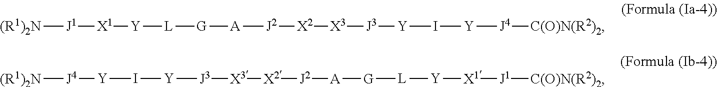
or a pharmaceutically acceptable salt thereof.

8. The oligopeptide or peptidomimetic of any one of claims 1 to 7, wherein at least two consecutive amino acid residues of X¹ and X^{1′} are selected from histidine (H), lysine (K), and arginine (R).

9. The oligopeptide or peptidomimetic of claims 1 to 8, wherein at least one of the amino acid residues of X¹ and X^{1′} is histidine (H).

10. The oligopeptide or peptidomimetic of any one of claims 1 to 9, wherein X¹ and X^{1′} consist of the following amino acid sequence: —H—H—I—.

11. The oligopeptide or peptidomimetic of any one of claims 1 to 6 and 8 to 10, wherein X² and X^{2′} are each an amino acid residue selected from glycine (G), alanine (A), valine (V), serine (S), and threonine (T).

12. The oligopeptide or peptidomimetic of any one of claims 1 to 6 and 8 to 10, wherein X² and X^{2′} are each valine (V).

13. The oligopeptide or peptidomimetic of any one of claims 1 to 12, wherein X² and X^{2′} are each an amino acid residue selected from glycine (G), alanine (A), serine (S), and threonine (T).

14. The oligopeptide or peptidomimetic of any one of claims 1 to 13, wherein X³ and X^{3′} are each asparagine (N).

15. The oligopeptide or peptidomimetic of any one of claims 2 to 14, wherein J¹, J², J³, and J⁴ are each absent.

16. The oligopeptide or peptidomimetic of any one of claims 2 to 15, wherein at least one of J, J², and J⁴ is a peptide linker region comprising at least one unnatural amino acid residue.

17. The oligopeptide or peptidomimetic of any one of claims 1 to 16, wherein each occurrence of R¹ and R² is hydrogen.

18. The oligopeptide or peptidomimetic of any one of claims 1 to 17, wherein the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

19. The oligopeptide or peptidomimetic of any one of claims 1 to 18, wherein at least one amino acid residue of Z¹, Z^{1′}, Z², Z^{2′}, Z³, Z^{3′}, Z⁴, and Z^{4′} is an unnatural amino acid residue.

20. The oligopeptide or peptidomimetic of any one of claims 1 to 19, wherein at least one amino acid residue of Z³, Z^{3′}, Z⁴, and Z^{4′} is an unnatural amino acid residue having a side chain comprising a substituted phenyl ring.

21. An oligopeptide or peptidomimetic of Formula (IIa) or Formula (IIb): 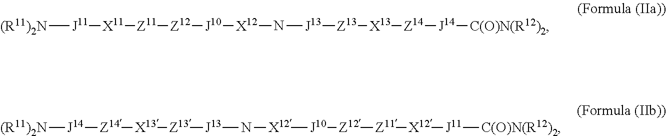
or a pharmaceutically acceptable salt thereof, wherein:
J¹⁰ is a linker region consisting of two to five amino acid residues;
J¹¹ and J¹⁴ and are each optional linker regions consisting of one to six amino acid residues;
J¹³ is an optional linker region consisting of one to three amino acid residues;
Z¹¹ and Z^{11′} are each an amino acid residue selected from: tyrosine (Y), phenylalanine (F), tryptophan (W), alanine (A), valine, (V), leucine (L), isoleucine (I), alloisoleucine, and methionine (M);
Z¹² and Z^{12′} are each an amino acid residue selected from: glycine (G), alanine (A), valine (V), leucine (L), isoleucine (I), and alloisoleucine;
Z¹³, Z¹⁴, Z^{13′}, and Z^{14′} are each an amino acid residue selected from histidine (H), tyrosine (Y) phenylalanine (F), and tryptophan (W);
X¹¹ and X^{11′} are each a tripeptide region consisting of three amino acid residues, wherein at least one amino acid residue is selected from histidine (H), lysine (K), arginine (R), alanine (A), cysteine (C), and methionine (M), and one amino acid residue is selected from leucine (L), isoleucine (I), methionine (M), and valine (V); provided that at most one of the three amino acid residues of X¹¹ and X^{11′} is histidine;
X¹² and X^{12′} are each an amino acid residue selected from glycine (G), alanine (A), valine (V), leucine (L), isoleucine (I), and methionine (M);
X¹³ and X^{13′} are each an amino acid residue selected from valine (V), leucine (L), isoleucine (I), alloisoleucine, methionine (M), phenylalanine (F), and tyrosine (Y);
R¹¹ is selected at each occurrence from hydrogen, —C(O)C₁₋₄ alkyl, and B—R¹³;
R¹² is selected at each occurrence from hydrogen and B—R¹³;
wherein B is absent or an organic linker, and R¹³ is a label or a water-soluble oligomer;
wherein the amino acids of X¹¹, X¹², Z¹¹, Z¹², Z¹³, and Z¹⁴ are L-amino acids, and the amino acids of X^{11′}, X^{12′}, Z^{11′}, Z^{12′}, Z^{13′}, and Z^{14′} are D-amino acids.

22. The oligopeptide or peptidomimetic of claim 21, wherein the oligopeptide or peptidomimetic is represented by Formula (IIa-1) or Formula (IIb-1): 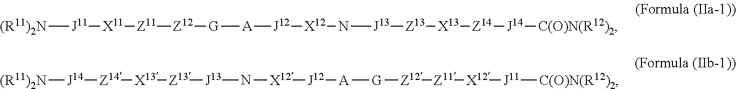
or a pharmaceutically acceptable salt thereof, wherein:
J¹² and J¹³ are each an optional linker region consisting of one to three amino acid residues, wherein J¹² and J¹³ comprise the same number of amino acid residues.

23. The oligopeptide or peptidomimetic of claim 21 or claim 22, wherein Z¹¹ and Z^{11′} are each an amino acid residue selected from: tyrosine (Y), phenylalanine (F), tryptophan (W).

24. The oligopeptide or peptidomimetic of any one of claims 21 to 23, wherein Z¹² and Z^{12′} are each leucine (L).

25. The oligopeptide or peptidomimetic of any one of claims 21 to 24, wherein the oligopeptide or peptidomimetic is represented by Formula (IIa-2) or Formula (IIb-2): 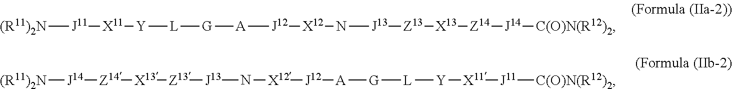
or a pharmaceutically acceptable salt thereof.

26. The oligopeptide or peptidomimetic of any one of claims 21 to 25, wherein Z¹³, Z¹⁴, Z^{13′}, and Z^{14′} are each an amino acid residue selected from tyrosine (Y) and phenylalanine (F).

27. The oligopeptide or peptidomimetic of any one of claims 21 to 26, X¹³ and X^{13′} are each an amino acid residue selected from leucine (L), isoleucine (I), and alloisoleucine.

28. The oligopeptide or peptidomimetic of any one of claims 21 to 27, wherein the oligopeptide or peptidomimetic is represented by Formula (IIa-3) or Formula (IIb-3): 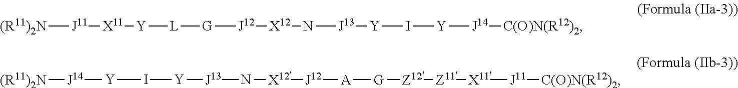
or pharmaceutically acceptable salt thereof.

29. The oligopeptide or peptidomimetic of any one of claims 21 to 28, wherein the oligopeptide or peptidomimetic is represented by Formula (IIa-4) or Formula (IIb-4): 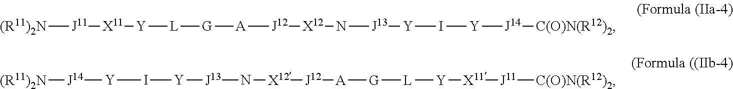
or pharmaceutically acceptable salt thereof.

30. The oligopeptide or peptidomimetic of any one of claims 21 to 29, wherein at least two consecutive amino acid residues of X¹¹ and X^{11′} are selected from histidine (H), lysine (K), and arginine (R), and one amino acid residue is isoleucine (I).

31. The oligopeptide or peptidomimetic of any one of claims 21 to 30, wherein at least one of the amino acid residues of X¹ and X^{1′} is histidine (H).

32. The oligopeptide or peptidomimetic of any one of claims 21 to 31, wherein X¹² and X^{12′} are each valine (V).

33. The oligopeptide or peptidomimetic of any one of claims 22 to 32, wherein J¹¹, J¹², J¹³, and J¹⁴ are each absent.

34. The oligopeptide or peptidomimetic of any one of claims 22 to 33, wherein at least one of J¹¹, J¹², J¹³, and J¹⁴ is present.

35. The oligopeptide or peptidomimetic of any one of claims 21 to 34, wherein each occurrence of R¹ and R² is hydrogen.

36. The oligopeptide or peptidomimetic of any one of claims 21 to 35, wherein the oligopeptide or peptidomimetic comprises at least one peptidomimetic amino acid residue.

37. The oligopeptide or peptidomimetic of claim 36, wherein the at least one peptidomimentic amino acid residue are independently selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

38. The oligopeptide or peptidomimetic of any one of claims 21 to 37, wherein at least one amino acid residue of Z¹¹, Z^{11′}, Z¹², Z^{12′}, Z¹³, Z^{13′}, Z¹⁴, and Z^{14′} is an unnatural amino acid residue.

39. The oligopeptide or peptidomimetic of any one of claims 21 to 38, wherein at least one amino acid residue of Z¹¹, Z^{11′}, Z¹³, Z^{13′}, Z¹⁴, and Z^{14′} is an unnatural amino acid residue having a side chain comprising a substituted phenyl ring.

40. The oligopeptide or peptidomimetic of any one of claims 21 to 39, wherein at least one occurrence of R¹¹ and R¹² is B—R¹³.

41. An oligopeptide or peptidomimetic of Formula (IIIa) or Formula (IIIb): 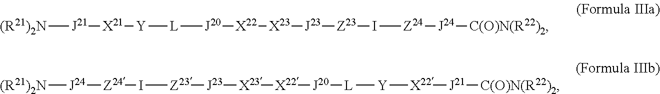
or pharmaceutically acceptable salt thereof, wherein:
J²⁰ is a linker region consisting of two to five amino acid residues;
J²¹ and J²⁴ are each optional linker regions consisting of one to six amino acid residues;
J²³ is an optional linker region consisting of one to three amino acid residues;
Z²³, Z²⁴, Z^{23′}, and Z^{24′} are each an amino acid residue selected from histidine (H), tyrosine (Y), phenylalanine (F), and tryptophan (W);
X²¹ and X^{21′} are each a tripeptide region consisting of one to six amino acid residues, each of which is independently selected from: histidine (H), lysine (K), arginine (R), alanine (A), cysteine (C), and methionine (M), and one amino acid residue is selected from leucine (L), isoleucine (I), methionine (M), and valine (V);
X²² and X^{22′} are each independently selected from glycine (G), alanine (A), valine (V), leucine (L), isoleucine (I), alloisoleucine, methionine (M), serine (S), and threonine (T);
X²³ and X^{23′} are each an amino acid residue selected from asparagine (N), glutamine (Q), serine (S), and threonine (T);
R²¹ is selected at each occurrence from hydrogen, —C(O)C₁₋₄ alkyl, and L-R²³;
R²² is selected at each occurrence from hydrogen and B—R²³, wherein at least one occurrence of
R²¹ or R²² is selected from —C(O)C₁₋₄ alkyl and B—R²³;
wherein B is absent or an organic linker, and R²³ is a label or a water-soluble oligomer;
wherein the amino acid residues of X²¹, X²², X²³, Z²¹, Z²², Z²³ and Z²⁴ are L-amino acid residues, and the amino acid residues of X^{21′}, X^{22′}, X^{23′}, Z^{21′}, Z^{22′}, Z^{23′} and Z^{24′} are D-amino acid residues.

42. The oligopeptide or peptidomimetic of claim 41, wherein the oligopeptide or peptidomimetic is represented by Formula (IIIa-1) or Formula (IIIb-1): 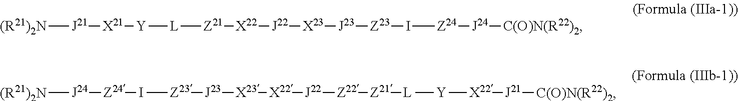
or pharmaceutically acceptable salt thereof, wherein
J²² and J²³ are each an optional linker region consisting of one to three amino acid residues, wherein J²² and J²³ comprise the same number of amino acid residues;
Z²¹, Z^{21′}, Z²² and Z^{22′} are each independently selected from: glycine (G), alanine (A), and serine (S), wherein the amino acid residues of Z²¹ and Z²² are L-amino acid residues, and the amino acid residues of Z^{21′} and Z^{22′} are D-amino acid residues.

43. The oligopeptide or peptidomimetic of claim 41, wherein J²¹, J²², J²³, and J²⁴ are each absent.

44. The oligopeptide or peptidomimetic of claim 41 to 42, wherein at least one of J²¹, J²², J²³, and J²⁴ is a peptide linker region comprising at least one unnatural amino acid residue.

45. The oligopeptide or peptidomimetic of any one of claims 41 to 43, wherein the oligopeptide or peptidomimetic comprises at least one peptidomimentic amino acid residue selected from a peptoid amino acid residue, an N-alkyl amino acid residue, a thioamide, a sulfonamide, a phosphonamide, an azapeptide, an ethylene, a hydroxylethylene, a hydroxylethylamine, a β-hydroxy-γ-amino acid residue, and a triazole.

46. The oligopeptide or peptidomimetic of any one of claims 41 to 44, wherein at least one amino acid residue of Z²¹, Z^{21′}, Z²², Z^{22′}, Z²³, Z^{23′}, Z²⁴, and Z^{24′} is an unnatural amino acid residue.

47. The oligopeptide or peptidomimetic of any one of claims 41 to 45, wherein at least one amino acid residue of Z²³, Z^{23′}, Z²⁴, and Z^{24′} is an unnatural amino acid residue having a side chain comprising a substituted phenyl ring.

48. The oligopeptide or peptidomimetic of any one of claims 41 to 46, wherein B is absent.

49. The oligopeptide or peptidomimetic of any one of claims 41 to 46, wherein B is an organic linker.

50. The oligopeptide or peptidomimetic of claim 48, wherein the organic linker of B comprises a C₁₋₁₀ alkyl chain.

51. The oligopeptide or peptidomimetic of any one of claims 41 to 49, wherein R²³ is a label.

52. The oligopeptide or peptidomimetic of claim 50, wherein the label of R²³ comprises a fluorophore.

53. The oligopeptide or peptidomimetic of claim 50, wherein the label of R²³ comprises biotin.

54. The oligopeptide or peptidomimetic of claim 50, wherein the label of R²³ comprises an azide or an alkyne.

55. The oligopeptide or peptidomimetic of any one of claims 41 to 49, wherein R²³ is a water-soluble oligomer.

56. The oligopeptide or peptidomimetic of claim 54, wherein the water-soluble oligomer of R²³ comprises polyethylene glycol.

57. A pharmaceutical composition comprising an oligopeptide or peptidomimetic of any one of claims 1 to 56 and a pharmaceutically acceptable excipient.

58. A method of inhibiting FasL-mediated cell death comprising administering to a subject in need thereof an oligopeptide or peptidomimetic of any one of claims 1 to 56 or a pharmaceutical composition of claim 57.

59. A method of preventing cell death comprising administering to a subject in need thereof an oligopeptide or peptidomimetic of any one of claims 1 to 56 or a pharmaceutical composition of claim 57.
